(19) United States
(12) Patent Application Publication
(10) Pub. No.: US 2021/0222130 A1
(43) Pub. Date: Jul. 22, 2021

(54) MODEL FOR IN-VITRO SIMULATION OF THE BEHAVIOUR OF DYSFUNCTIONAL VESSELS

(71) Applicant: 1LAB SA, Manno (CH)

(72) Inventors: Enrico PERFLER, Manno (CH); Andrea BIFFI, Manno (CH); Stefano FARINA, Manno (CH)

(21) Appl. No.: 17/259,898

(22) PCT Filed: Aug. 5, 2019
(86) PCT No.: PCT/IB2019/056652

§ 371 Date: Jan. 12, 2021

(30) Foreign Application Priority Data
Aug. 7, 2018 (IT) 102018000007946

(51) Int. Cl. C12N 5/071; G01N 33/50; C12N 5/077; C12N 5/00; A61L 27/38; A61L 27/50

(52) U.S. Cl. CPC C12N 5/0691; A61L 2430/20; G01N 33/5061; G01N 33/5082; C12N 5/0697; C12N 5/0661; C12N 5/0062; A61L 27/3808; A61L 27/3826; A61L 27/507; C12N 2513/00; C12N 2533/50; C12N 2500/84; C12N 2500/40; C12N 2500/20; C12N 2500/14; C12N 2500/38; G01N 33/5064

(57) ABSTRACT

The present invention refers to a model for in-vitro simulation of the behaviour of dysfunctional human vessels, such as for example vessels affected by aneurysm, stenosis or sclerosis plaques, as an instrument for testing medical devices and drugs with the aim of verifying effectiveness and safety thereof prior to use thereof on humans. Specifically, the present invention refers to an in vitro model of a substantially tubular-shaped vascular structure having dysfunctional anatomical and physiological characteristics simulating the same vascular structure of a healthy subject whose vascular structure has been damaged or deformed or deteriorated due to a damage selected from among the group comprising or, alternatively, consisting of aneurysm, stenosis, sclerosis plaques, forms of tumours or cardiomyopathies having the characteristics as claimed in the attached claims. Furthermore, the present invention also refers to a reliable and reproducible industrialisation process for eliminating air bubbles for producing an engineered vascular tissue for the in vitro test of medicinal products for human use and veterinarian products for animal use.

The present invention refers to a model for in-vitro simulation of the behaviour of dysfunctional human vessels, such as for example vessels affected by aneurysm, stenosis or sclerosis plaques, as an instrument for testing medical devices and drugs with the aim of verifying the effectiveness and safety thereof prior to use thereof on humans. Specifically, the present invention refers to an in vitro model of a vascular structure, preferably a substantially tubular-shaped synthetic vascular structure having dysfunctional anatomical and physiological characteristics, simulating the vascular structure of a healthy subject whose vascular structure has been damaged or deformed or deteriorated due to a damage selected from among the group comprising or, alternatively, consisting of aneurysm, stenosis, sclerosis plaques, forms of tumours or cardiomyopathies having the characteristics as claimed in the attached claims. Furthermore, the present invention also refers to a reliable and reproducible industrialisation process for eliminating air bubbles for producing an engineered vascular tissue for the in vitro test of medicinal products for human use and veterinarian products for animal use.

It is known that the development of a medicinal product entails a long and sensitive process. Basically, in the medicinal or veterinarian product development process, whether a drug or medical device, prior to using the product in humans or in animals it is important to be able to determine the type of effects on the tissue/s with which it comes into contact so as to evaluate the aspects relating both to biological safety and to the efficiency of the medicinal product and foretell potential problems relating to the use thereof. In this context, the evaluation of the biological safety and efficiency of the medicinal product is extensive and complex while the evaluation of the interaction with the tissue/s of only one constituent material cannot be considered as isolated from the overall planning of the medicinal or veterinarian product which must be evaluated as a whole and in a context that can reproduce the conditions of use as faithfully as possible.

Today, the evaluation of biological safety and of the efficiency of a medicinal or veterinarian product are based on in vitro, ex vivo tests and on animal models that have significant differences with respect to the final conditions of use. In particular, the animal models have significant physiological differences that complicate the transposition of the validity of the results to humans. Such differences are observable even further when evaluating medicinal products for human use or veterinarian products for animal use that provide for the use thereof in the cardiovascular and peripheral vascular region, such as, for example, heart valves, stents, grafts, catheters, bandages and nets.

Unfortunately, the evaluation of the interaction of medicinal products for human use or veterinarian products for animal use with the vascular tissues and with the blood so as to be able to establish the biological safety and efficiency is crucial and the models currently in use reveal major limits and drawbacks both anatomical/structural and regarding the blood composition.

Furthermore, tests on animals have been at the centre of an intense and controversial debate around the issue whether using animals for biomedical testing can be considered morally acceptable. This issue led to the 3R principle already back in 1959, and it still remains a hotly disputed current topic in debates and in the international research programmes. The 3R principle refers to three key concepts: replacement, reduction and refinement. The 3R principle argues that research in the biomedical industry should aim at, with utmost effort possible, replacing or substituting the animal model with an alternative model; reducing the number of animals used in a given experimental protocol as much as possible; refining, i.e. improving the experimental conditions to which the animals are subjected.

The vascular tissue engineering industry shows that it is crucial to be able to produce a continuous (i.e. having a monolayer of confluent cells) and functional endothelium mainly consisting of endothelial cells (ECs).

The production of continuous and functional endothelium is a crucial factor towards promoting scaffold endothelisation and guaranteeing appropriate efficiency of the tissue-engineered constructs (consisting of scaffolds and cells), including preventing thrombosis and stenosis once said constructs have been implanted.

The in vitro generation of vascular endothelium or engineered vascular construct/tissue, in short provides for the following steps:
(1) seeding endothelial cells in the lumen of the scaffold and ensuing adhesion thereof,
(2) cell growth/proliferation and organisation as a function of mechanical stimuli (such as for example the flow of a fluid) to which they are subjected, and
(3) generating one or more layers of functional endothelial cells.

Due to the crucial importance of the seeding step, many research groups at university level have invested in research over the last decades creating various techniques for uniformly seeding the endothelial cells in the lumen of the scaffold and enhancing the seeding effectiveness. However, the results achieved have not been entirely satisfactory.

Static seeding and dynamic seeding currently represent the main endothelial cell seeding methods.

The simplest static method consists in pipetting a cell suspension directly on the luminal surface of the scaffold followed by a short incubation step on a Petri dish. This method strongly depends on the operator and it is complicated by the difficulty to obtain a uniform endothelial layer.

Vastly based on rotation, vacuum, electrostatic or magnetic forces, the dynamic methods on the contrary increase cell seeding effectiveness, uniformity and adhesion. Some of these dynamic principles could be directly transferred and paired with perfusion bioreactors, allowing a reduction in the handling of the scaffold. In this case, the scaffolds can be immediately seeded once housed in the bioreactor chamber.

Other research groups instead use the dynamic seeding method through a continuous injection of a cell suspension using a syringe pump, or through a continuous perfusion of the lumen of the scaffold seeded with a growth medium using a peristaltic pump, after injecting the endothelial cell suspension. These methods require higher volumes of cell suspension with respect to a pipetting/rotation seeding method due to the need of also filling the volume of the perfusion piping as well as the volume of the injection syringe, revealing possible limitations with reference to reducing costs (growth cells and media).

A completely different technique with respect to the ones described previously is based on dripping the cell suspension in the lumen of a scaffold. In this case, the main drawback lies in a low initial adhesion of the cells and a low reproducibility of the method given that it strongly depends on the operator.

Furthermore, the choice of a method for connecting a perfusion circuit (perfusion method), required for the perfusion of a scaffold, mainly tubular, to the bioreactor-scaffold system must be adapted to the experimental setup.

Considering the landscape regarding the methods for seeding and connecting a perfusion circuit (perfusion method) mentioned above, considerable limits and drawbacks still exist.

Therefore, there clearly arises the need to provide a process comprising a method for seeding and connecting the perfusion circuit (perfusion method) to the bioreactor-scaffold system that is reproducible, reliable and effective, especially considering the applicability of said process for GLP (Good Laboratory Practice)-approved Tissue engineering laboratories whose objective focuses on advanced preclinical tests and clinical tests.

There arises the need to develop a process for the production of engineered vascular tissues/constructs having a continuous (i.e. having a monolayer of confluent cells) and functional endothelium, wherein said process is simple, quick, effective, independent from the operator, well defined, highly reproducible and reliable, comprising: (1) a method for seeding cells, mainly endothelial cells, in the lumen of a scaffold, that guarantees the homogeneity and the uniform adhesion of the endothelial cells, with the elimination of air bubbles in the scaffold; (2) a method for connecting the perfusion circuit to the bioreactor-scaffold system (perfusion method) capable of guaranteeing sterility and maintaining the absence of air bubbles in the assembled system consisting of the perfusion circuit and bioreactor-scaffold system.

There also arises the need of being able to develop in vitro engineered vascular tissues/constructs comprising a continuous and functional endothelium for conducting advanced preclinical and clinical tests, so as to avoid using laboratory cavies to conduct preclinical and clinical tests. Therefore, there arises the need for a procedure that is reliable, effective and reproducible for the industrialisation of said in vitro engineered vascular tissues/constructs.

As observed above, the process for developing and approving medicinal products, such as for example medical devices and drugs, normally requires conducting numerous preclinical tests to be conducted in-vitro, ex-vivo and in-vivo on animals prior to conducting clinical testing on humans. Despite animal testing offering an in vivo model for the test of medicinal products, the anatomical structure, the physiology and the blood composition considerably differ from those of humans and the results achieved on the animal model may considerably differ from those achieved in human testing.

Furthermore, besides being technically complex, causing diseases (such as for example vascular dysfunctions, aneurysms or stenosis) using animal models is widely ethically inadmissible. Thus, the in vivo test on animals cannot be conducted to verify whether the medicinal product is effective at treating given diseases. To confirm the scientific willingness and policy towards minimising in vivo testing on animals, since 1987 the 3R (Replace, Reduce, Refine) Research Foundation has been promoting the development of methods alternative to the animal model and has been creating awareness among the public and scientific community around animal testing only in the absence of alternative methods so as to meet the “technical” requirement, with the aim of reducing the number of laboratory animals and suffering inflicted on the animals to the minimum. Methods alternative to animal testing represent significant progress not only from an ethical point of view as regards treating animals more responsibly but above all as concerns the possibility of developing models that are reliable, reproducible and that allow to simulate the “human” environment to the uttermost. Computerised simulations and in vitro cell culture for the preliminary analysis of some characteristics of medicinal products have been created in recent years to this end.

In compliance with the 3R principle and with the aim of overcoming the anatomical/physiological limitations of animal models, it would be useful to have dysfunctional engineered vascular models capable of reproducing the diseases (such as for example stenosis, aneurysms) and “special” vascular structures as testing platform for medical devices and drugs prior to the use thereof in humans. The use of an in vitro system capable of replicating, in a standardised manner, human anatomical structures and the in-vivo environment would not only allow to accelerate the research and development of new medicinal products, but also to obtain more reliable responses on the safety and on the effectiveness of the tested products with respect to the animal model.

The Applicant met the aforementioned needs.

Forming an object of the present invention is an in vitro model of a substantially tubular-shaped vascular structure having dysfunctional anatomical and physiological characteristics, simulating the vascular structure of a healthy subject whose vascular structure has been damaged or deformed or deteriorated due to a damage selected from among the group comprising or, alternatively, consisting of aneurysm, stenosis, sclerosis plaques, forms of tumours or cardiomyopathies having the characteristics as claimed in the attached claims.

Furthermore, forming an object of the present invention is an in vitro model of a substantially tubular-shaped vascular structure having dysfunctional anatomical and physiological characteristics simulating the same vascular structure of a healthy subject whose vascular structure has been damaged or deformed or deteriorated due to a damage selected from among the group comprising or, alternatively, consisting of aneurysm, stenosis, sclerosis plaques, forms of tumours or cardiomyopathies, wherein said model comprises or, alternatively, consists of one or more biocompatible porous polymeric supports (“scaffolds”) capable of promoting a cell adhesion and growth, wherein said scaffold is seeded with endothelial cells that cover a lumen of the scaffold and constitute an endothelium having a monolayer of confluent cells, said scaffold being provided with deformities or defects on a tubular structure thereof, having the characteristics as claimed in the attached claims.

Preferably said in vitro model has a vascular structure that was selected from among the blood vessels or valves of the central or peripheral circulatory system; preferably arteries, veins, capillaries, aortic or mitral valve. Preferably, said in vitro model is a dysfunctional vascular model. Preferably, said vascular structure is synthetic vascular structure. Preferably, said scaffold consists of electrospun silk fibroin, copolymers of polyglycolic acid/polylactic acid (PGA/PLA) or copolymers of polyglycolic acid/polycaprolactone (PGA/PCL). Preferably, said in vitro model comprises or, alternatively, consists of one or more scaffolds seeded with endothelial cells, and optionally muscle cells. Preferably, said deformities or said defects of the tubular structure comprise bifurcations, curvatures, elbows, constrictions, dilatations or combinations thereof.

Forming an object of the present invention is a method for testing a medical device or a drug with the aim of verifying the effectiveness and safety thereof prior to the in vivo use thereof on humans or animals, said method comprising the following steps:
preparing a substantially tubular-shaped scaffold having the dysfunctional anatomical and physiological characteristics suitable to simulate a damage or a deformation or a deterioration due to an aneurysm, stenosis, sclerosis plaques, forms of tumours or cardiomyopathies;
seeding at least part of the internal lumen of said scaffold with endothelial cell lines so as to obtain a continuous and homogeneous layer of seeded endothelial cells (seeding method), optionally seeding at least part of the outer surface of said scaffold with muscle cell lines;
promoting the growth of said endothelial cells, and optionally said muscle cells, up to obtaining a continuous and uniform layer of endothelial cells, to obtain said in vitro model;
introducing into said in vitro model a medical device or a drug subject of test, and
allowing the circulation (perfusion model) in said in-vitro model comprising said medical device or drug of a human whole blood sample, artificial blood or derivatives thereof so as to evaluate the behaviour and the interaction of said medical device or drug with said human whole blood sample, artificial blood or derivatives thereof.

Forming an object of the present invention is a model for in-vitro simulation of the behaviour of dysfunctional human vessels comprising or, alternatively, consisting of vessels affected by aneurysm, stenosis or sclerosis plaques, as an instrument for testing medical devices and drugs with the aim of verifying the effectiveness and safety thereof prior to use thereof on humans, having the characteristics as claimed in the attached claims.

Also forming an object of the present invention is providing in vitro vascular structure models, mainly substantially tubular-shaped, having, depending on the need, dysfunctional anatomical and physiological characteristics with respect to the healthy human vascular structure, such as, by way of non-limiting example, aneurysms, stenosis, sclerosis plaques, having the characteristics as claimed in the attached claims.

Forming another object of the present invention are dysfunctional vascular models comprising or, alternatively, consisting of one or more scaffolds seeded with suitable selected cells, having the characteristics as claimed in the attached claims.

Forming another object of the present invention is a method for providing said dysfunctional vascular models using scaffolds seeded with suitable selected cells, having the characteristics as claimed in the attached claims.

Such dysfunctional vascular structures include: (i) a scaffold consisting of biocompatible material suitable to be electrospun, such as for example silk fibroin, or molten in moulds or printed using bioprinters such as for example polylactic acid, polycaprolactone, etc. The structure of the scaffold is suitable to allow the seeding of endothelial vascular cells in the scaffold and to allow the through-flow of fluids such as growth medium for sustaining the cells, blood (artificial or whole human).

A different embodiment of the structure of the scaffold described in the previous point provides for the possibility of seeding, on the outer surface of the scaffold, as a function of the characteristics to be tested in the medicinal product subject of the test, muscle cells or nervous cells, providing for a weft that promotes the orientation of said cells so as to be able to simulate the vasoconstriction or vasodilation depending on the mechanical, pharmacological or chemical stimulus to which the model is subjected.

In the scaffold, for example in order to simulate clots, sclerosis plaques, thrombosis or stenosis, blood components (such as for example platelets, cholesterol, erythrocytes, etc.).

Still with the aim of simulating a dysfunction of the vascular structure, the scaffolds can be made having deformities or defects on the tubular structure thereof, such as for example, bifurcations, curvatures, elbows, constrictions, dilatations.

The hydraulic circuit and the bioreactor into which the scaffold is inserted is outlined hereinafter in the present description to which reference shall be made with the help of the attached drawings. The circuit, already as provided, allows the perfusion of the seeded cells on the scaffold with growth medium so as to allow the survival and growth thereof, with the possibility of passing to a pulsatile regime upon replacing the growth medium with blood (whole or diluted with growth medium or with eluate of the product to be tested), or with artificial blood, or with growth medium diluted with eluate of the product to be tested. The circuit allows—by means of suitable valve upstream of the bioreactor—to introduce the medicinal product (drug, medical device) into the scaffold.

The dysfunctional scaffolds subject of the present invention and obtained by means of the method described herein were tested using:
Blood analysis (blood alterations caused by the dysfunction of the vessel and/or by the product used for treating the dysfunction of the vessel) or hemocompatibility of the medicinal product.
Biocompatibility of the medicinal product used for treating vascular dysfunction such as for example stents, devices for the embolization of the aneurysms, catheters, heart valves, drugs, etc.
Verifying functionality, feasibility, or appropriateness of the design of the medicinal product at correcting the vascular dysfunction.

Furthermore, after a long and intense research and development activity, the Applicant created a process comprising a seeding method and a method for connecting the perfusion circuit to the bioreactor-scaffold system, having the characteristics as claimed in the attached claims.

Preferred embodiments of the present invention will be clear from the detailed description that follows.

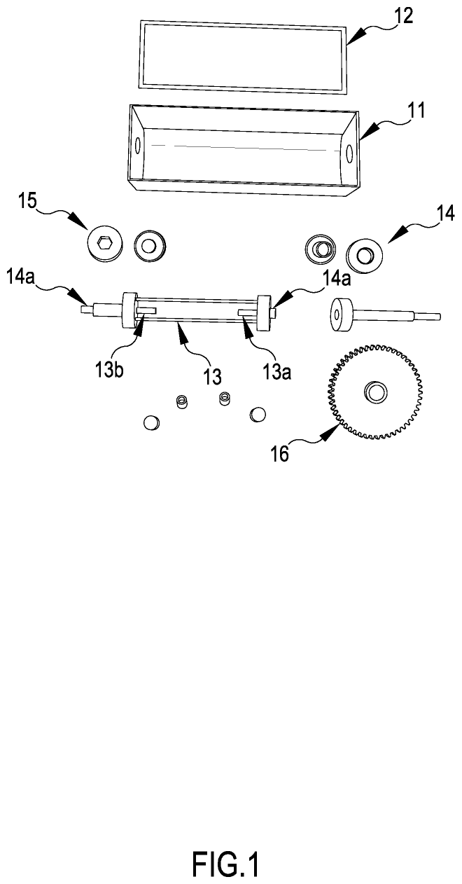

FIGS. 1-27 are outlined hereinafter in the present description.

In the context of the present invention the expression continuous and functional endothelium is used to indicate an endothelium, with physiological-like behaviour, wherein the endothelial cells are adjacent to each other, adhered to the scaffold and expressing markers typical of the endothelial cells, such as for example Von Willebrand factor (VWF), cluster of differentiation 31 (CD31), vascular cell adhesion molecule 1(VCAM-1). In particular, the expression continuous endothelium is used to indicate an endothelium having a monolayer of confluent cells.

In the context of the present invention the expression scaffold is used to indicate a biocompatible porous polymeric medium capable of promoting the cell adhesion and growth, endothelial cells in this case. The polymeric scaffold can be of synthetic or natural origin and consist of only one polymer or copolymers (entirety of polymers), such as for example electrospun silk fibroin or copolymers of PGA/PLA (polyglycolic acid/polylactic acid) or PGA/PCL (polyglycolic acid/polycaprolactone).

In the context of the present invention the expression “confluence” refers to a surface of the scaffold (in particular an inner surface or lumen of such scaffold) which is covered by adherent cells. In particular, so-called “confluent cells” have a confluence equivalent to or greater than 90%, preferably comprised between 90% and 100%, even more preferably comprised between 95% and 100%, hence substantially the entire surface of the scaffold is covered by adherent cells and there is no more surface left available on the scaffold so that the cells can grow as a monolayer.

In the context of the present invention the cells constituting an endothelium of a vascular tissue are defined as endothelial cells. Examples of endothelial cells are the HAOECs (human aortic endothelial cells), HCAECs (human coronary artery endothelial cells), HMEVECs (human dermal microvascular endothelial cells), or HUVECs (human umbilical vein endothelial cells).

In the context of the present invention, a scaffold having the lumen mainly covered by functional endothelial cells following the in vitro endothelisation process is defined as engineered vascular construct.

Said endothelial cells covering the lumen of the scaffold constitute a continuous endothelium, i.e. an endothelium having a monolayer of confluent cells.

In the context of the present invention, the cell growth and maintenance fluid, specific for each type of cell, is defined as growth medium. In particular, as concerns endothelial cells used in this specific case (HUVECs—Human Umbilical Vein Endothelial Cells, purchased from Sigma Aldrich, code 200-05n), the growth medium is Endothelial Growth Medium (EGM, Sigma Aldrich, 211-500). EGM contains fetal bovine serum (2%), adenine (0.2 μg/ml), ammonium metavanadate (0.0006 μg/ml), amphotericin B (0.3 μg/ml), calcium chloride 2H₂O (300 μg/ml), choline chloride (20 μg/ml), copper sulphate 5H₂O (0.002 μg/ml), trioptic acid DL-6,8 (0.003 μg/ml), folinic acid (calcium) (0.6 μg/ml), heparin (4 μg/ml), hydrocortisone (2 μg/ml), L-aspartic acid (15 μg/ml), L-cysteine (30 μg/ml), L-tyrosine (20 μg/ml), manganese sulphate monohydrate (0.0002 μg/ml), ammonium molybdate 4H₂O (0.004 μg/ml), nicotinamide (8 μg/ml), nickel chloride 6H₂O (0.0001 μg/ml), penicillin (60 μg/ml), phenol red sodium salt (15 μg/ml), potassium chloride (300 μg/ml), putrescine dihydrochloride (0.0002 μg/ml), pyridoxine hydrochloride (3 μg/ml), sodium metasilicate 9H₂O (3 μg/ml), sodium sulphate 7H₂O (200 μg/ml), sodium selenite (0.01 μg/ml), streptomycin sulphate 100 μg/ml), thiamine hydrochloride (4 μg/ml), and zinc sulphate 7H₂O (0.0003 μg/ml). The fresh growth medium is the sterile medium not used previously, directly supplied by the manufacturer. The expression hot growth medium is used to indicate that the growth medium was previously heated at a temperature comprised in the range between 30° C. and 45° C., preferably at 37° C.

The process subject of the present invention comprises a seeding method and a method for connection between a bioreactor and a scaffold perfusion circuit (perfusion method), preferably tubular scaffolds, for engineering a vascular tissue with ensuing production of vascular grafts engineered (vascular constructs/tissues) for testing medicinal products. Said process, comprising the seeding method and the method for connecting a perfusion circuit for a bioreactor-scaffold system (perfusion method), advantageously guarantees the accurate removal of air bubbles from the system described hereinafter and, thus, it guarantees maximum reproducibility of the process. Furthermore, reducing the risk of the air bubbles coming into contact with the endothelial cells allows to prevent damaging the endothelial cells and it allows to obtain a confluent monolayer of endothelial cells adhered onto the lumen of the scaffold (continuous and functional endothelium). In this specific case, such seeding method and method for connecting a scaffold perfusion circuit (perfusion method) is applied onto a scaffold preferably tubular electrospun silk fibroin in a bioreactor for the perfusion.

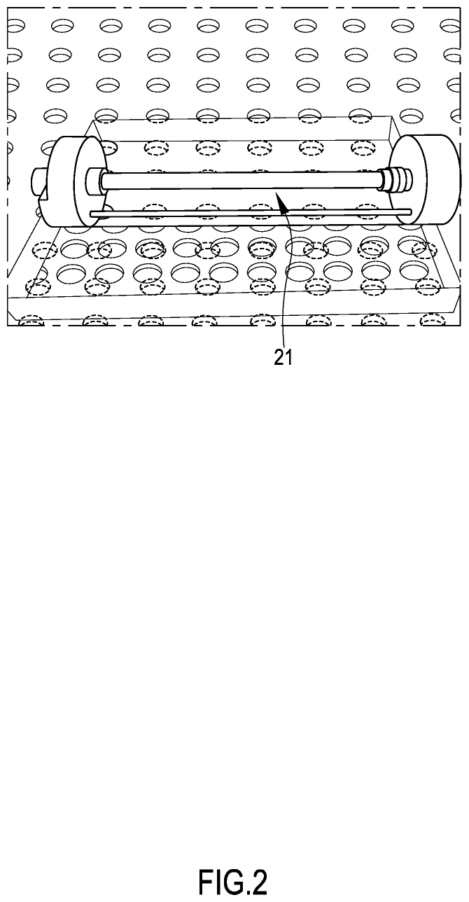

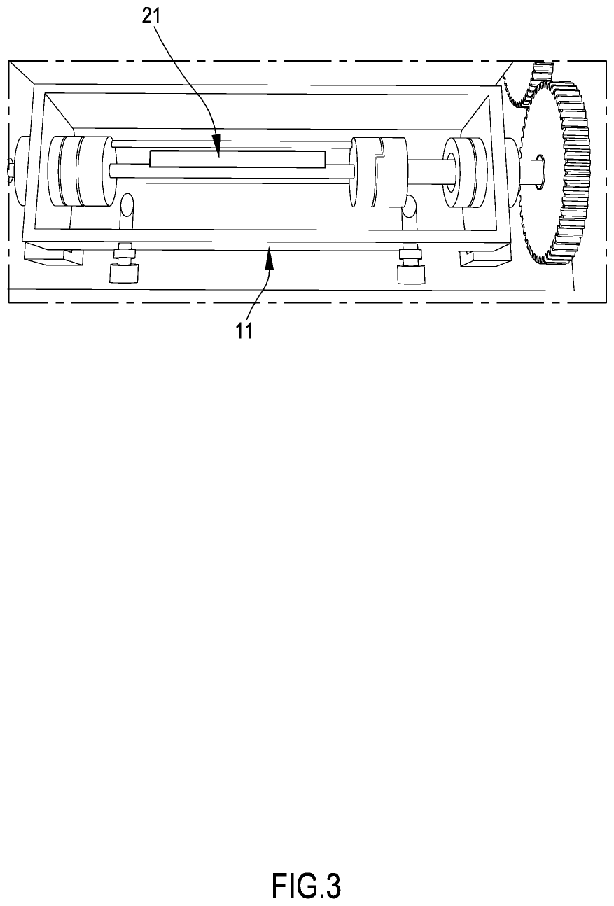

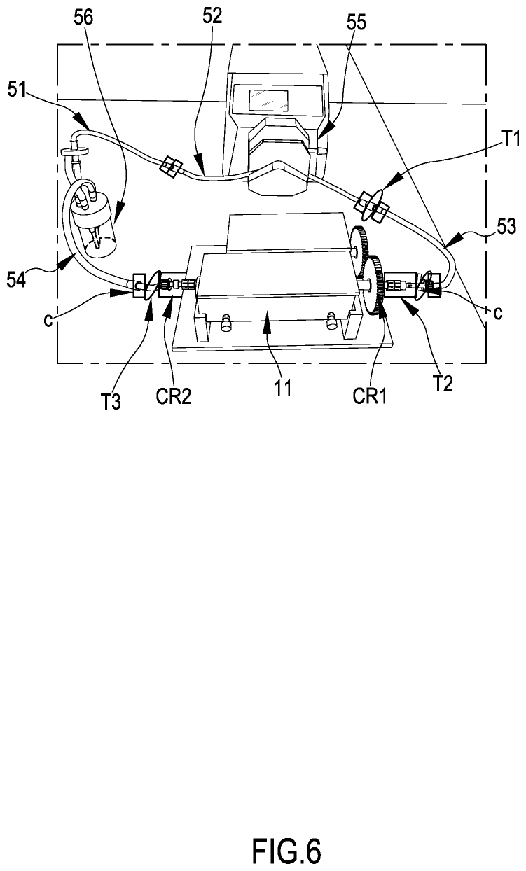

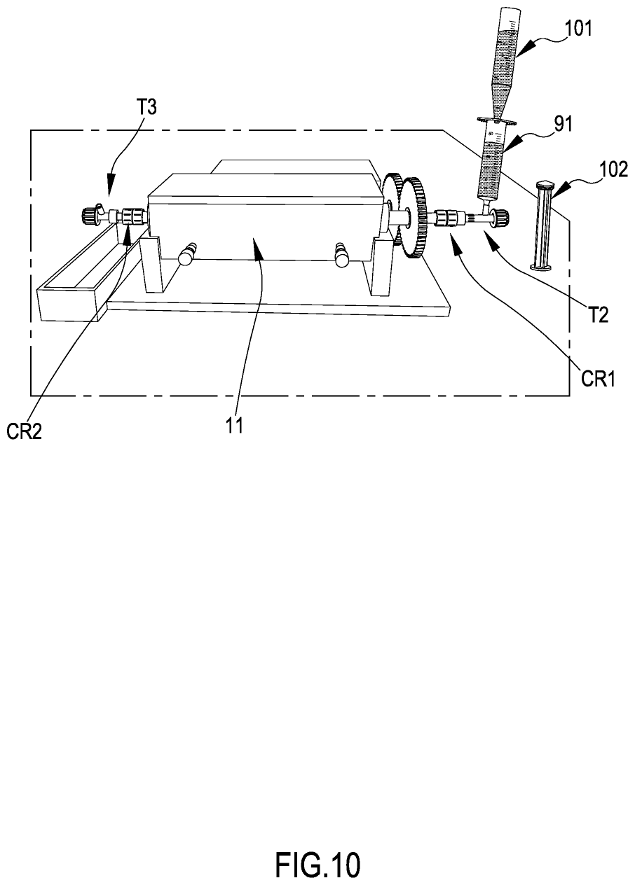

The process of the present invention allows to overcome the limitations of the models currently available and meeting the 3R requirements in that it offers a valid alternative to using animal models. Forming an object of the present invention is a process for producing an engineered vascular tissue or construct, preferably a scaffold (FIG. 2, 21) having a lumen covered with a functional and continuous endothelium having a confluent cell monolayer, preferably usable for testing medicinal or veterinarian products wherein said process comprises applying:
a method for seeding an endothelial cell culture in the lumen of a scaffold (21) to obtain a seeded scaffold (21); said seeded scaffold (21) being present in a bioreactor (11), to obtain a bioreactor (FIG. 3, 11)-seeded scaffold (21) system;
   wherein said seeding method comprises the steps of:
releasing said endothelial cell culture in form of a cell suspension comprising a fresh growth medium and endothelial cells in a container (FIG. 10, 91) mounted on a T-shaped connector (FIG. 10, T2) arranged upstream of the bioreactor (11) by means of a rotary connector (FIG. 10, CR1); followed by
releasing said endothelial cell culture in the lumen of the scaffold (21) present in the bioreactor chamber (11) with a continuous flow such that the flow speed allows said cell suspension to drip into the T-shaped connector (T2) without generating air bubbles and pushing the air bubbles present in the lumen of the scaffold (21) towards an opening of a T-shaped connector (FIG. 10, T3) arranged downstream of the bioreactor (11) allowing the outflow thereof;
   and, subsequently,
a method for perfusion—with a fresh growth medium having a temperature comprised in the range between 30° C. and 45° C., preferably at 37° C.—of the endothelial cells present in the lumen of said seeded scaffold (21); said perfusion method being obtained by connecting a perfusion circuit (FIG. 6; 51, 52, 53, 54, 55, and 51-56 or 51-57 and BT) to said bioreactor (11)-seeded scaffold (21) system;
   wherein said perfusion method comprises a step of
partly filling an element for removing the air bubbles (71 or BT) present in the perfusion circuit with said fresh growth medium, wherein said element for removing the air bubbles (71 or BT) comprises a chamber, a cap that closes said chamber, an access with inflow function (211) and an access with outflow function (212), wherein said chamber of the element for removing the air bubbles (71 or BT) has a volume and wherein a first part of said volume is filled with said fresh growth medium and wherein a second part of said volume is filled with air, said second part of said volume having the function of trapping the air bubbles present in said fresh growth medium which flows through said access with inflow function (211) and said access with outflow function (212).

With the aim of illustrating preferred embodiments, the proposed technical solution represented by the process subject of the present invention is, for ease of comprehension, divided into the two methods which are described in detail separately hereinafter: (1) method for seeding a cell culture in the lumen of a scaffold, preferably a tubular scaffold; (2) method for connecting a perfusion circuit to a bioreactor-scaffold system (perfusion method).

### (1) Method for Seeding a Cell Culture in the Lumen of a Scaffold According to a Preferred Embodiment.

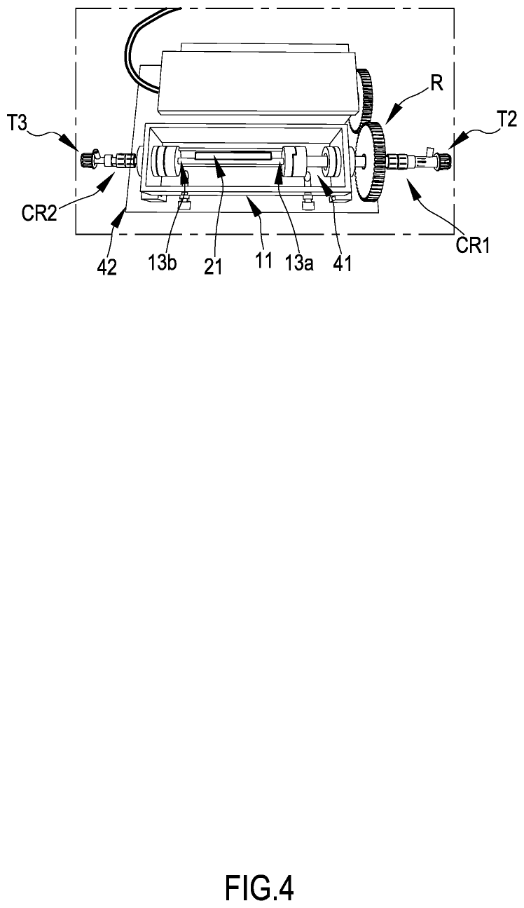

The method for uniform seeding of endothelial cells for a bioreactor-scaffold system, comprises a plurality of steps carried out sequentially and under sterile conditions:
1.1 A scaffold (FIG. 2; 21), preferably a tubular scaffold, for example an electrospun silk fibroin tubular scaffold, mounted on the grips of the scaffold-holder (FIG. 1; 13, 13a, 13b) is housed in the bioreactor chamber, to obtain a bioreactor-scaffold system. Applied at both ends of the bioreactor (upstream and downstream) are rotary connectors (FIG. 4; CR1, CR2) and T-shaped connectors (FIG. 4; T2, T3). In the context of the present invention the expression bioreactor-scaffold system is used to indicate the assembly of the bioreactor and the scaffold (FIG. 3; 11, 21), preferably a tubular scaffold such as for example an electrospun silk fibroin tubular scaffold, which is housed and fixed by means of grips of the scaffold-holder(FIG. 1; 13, 13a, 13b) which is arranged in the bioreactor. The scaffold, preferably tubular, is fastened to the grips of the scaffold-holder (which is internally hollow so as to allow the perfusion of the scaffold) with self-fastening strips, after having protected the scaffold, an electrospun silk fibroin in this case, with a sterilised teflon tape.

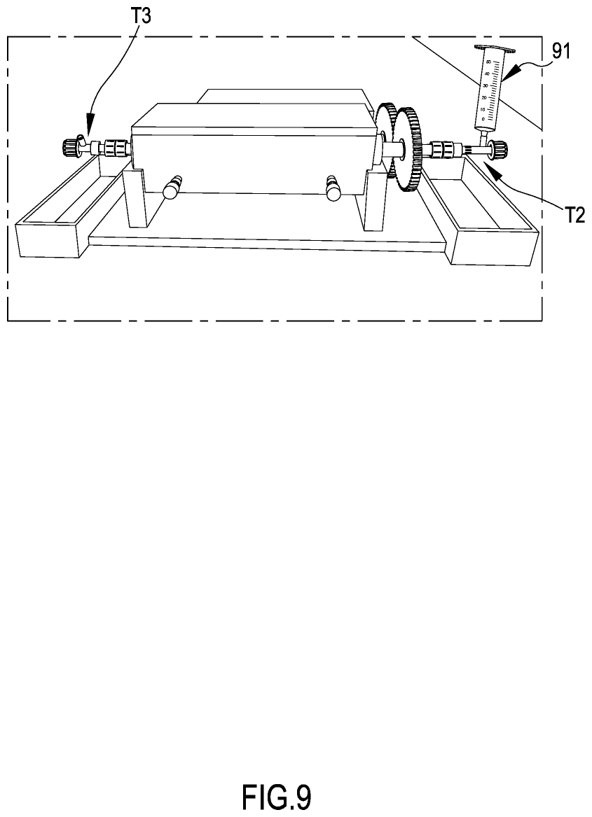

The insertion of the scaffold-holder 13 into the bioreactor 11 occurs in a manner such that the inlet upstream of the bioreactor coincides (FIG. 4; CR1, 41) with an end of the scaffold (FIG. 4; 13a) and the opening downstream of the bioreactor chamber (FIG. 4; CR2, 42) coincides with the other end of the scaffold (FIG. 4; 13b). In this manner, the scaffold 21 is perfectly coaxial with respect to the perfusion path generated by the internally hollow scaffold-holder. The larger axis according to which the scaffold mounted in the bioreactor is oriented is defined as the longitudinal axis.
1.2 The scaffold is preconditioned using fresh growth medium injected into the lumen of the scaffold which is housed in the bioreactor, using a syringe with a luer-lock connector which is engaged to one of the two ends of the bioreactor by means of a T-shaped connector (FIG. 9; T2). Subsequently, the open ends of the connectors arranged upstream and downstream of the bioreactor are closed using caps so as to avoid the emptying of the lumen of the scaffold. Furthermore, the fresh growth medium is inserted into the bioreactor chamber until the scaffold housed therein is fully covered. In this manner, the scaffold housed in the bioreactor chamber is preconditioned, preferably for 1 hour to 25° C., using a fresh growth medium both internally (in the lumen) and externally.
1.3 After preconditioning, the scaffold inside the bioreactor preconditioned with the growth medium injected into the lumen is emptied preferably using a sterile pipette and the growth medium previously introduced into the chamber is eliminated preferably using a sterile pipette. The growth medium residues present in the connectors (rotated and T-shaped) arranged downstream and upstream of the bioreactor are eliminated with a vacuum using a pipette, without making the scaffold collapse.
1.4 The T-shaped connectors arranged upstream (FIG. 9, T2) and downstream (FIG. 9; T3) of the end of the bioreactor, with the scaffold therein mounted on the grips, are directed with the upper opening upwards (at 90° with respect to the plane in which the bioreactor-scaffold system lies). Subsequently, the lateral opening of the T-shaped connectors arranged downstream (T3) and upstream (T2) of the bioreactor is capped. Mounted on the opening facing upwards of the T-shaped connector (FIG. 9; T2) arranged upstream of the bioreactor is a container, preferably a syringe (FIG. 9; 91) with a luer-lock connector with capacity for example of 5 ml without the plunger thereof. The opening of the T-shaped connector (FIG. 9; T3) arranged upstream of the bioreactor remains open instead (FIG. 9).
1.5 Using a pipette, preferably a sterile plastic pipette with capacity of for example 25 ml (FIG. 10; 101), is drawn from a container prepared in which is a cell suspension consisting of fresh growth medium and endothelial cells (e.g. HUVECs). Subsequently, the drawn cell suspension is released into the container or into the syringe (FIG. 10; 91) mounted on the T-shaped connector element (FIG. 10; T2) upstream of the bioreactor through the element (FIG. 10; CR1). The cell suspension must be released, using the pipette (FIG. 10; 101), with capacity of for example 25 ml, with a continuous flow so that the flow speed allows the cell suspension to drip into the T-shaped connector (FIG. 10; T2) without generating air bubbles and pushing possible air bubbles present in the scaffold towards the opening of the T-shaped connector T3 arranged downstream (FIG. 10; T3) of the bioreactor 11 and, hence flow out (FIG. 10).
1.6 When the cell suspension, loaded using a syringe, reaches the open end of the T-shaped connector

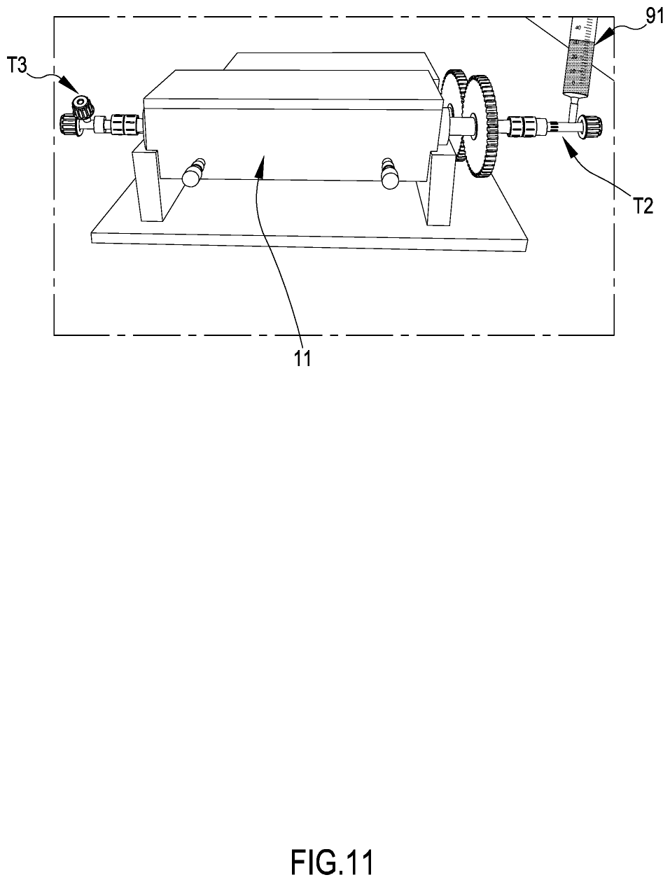

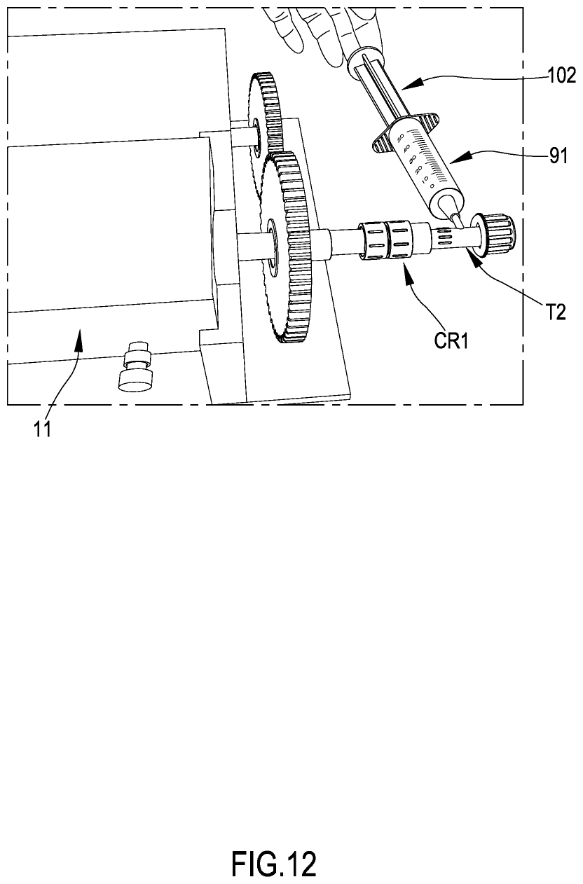

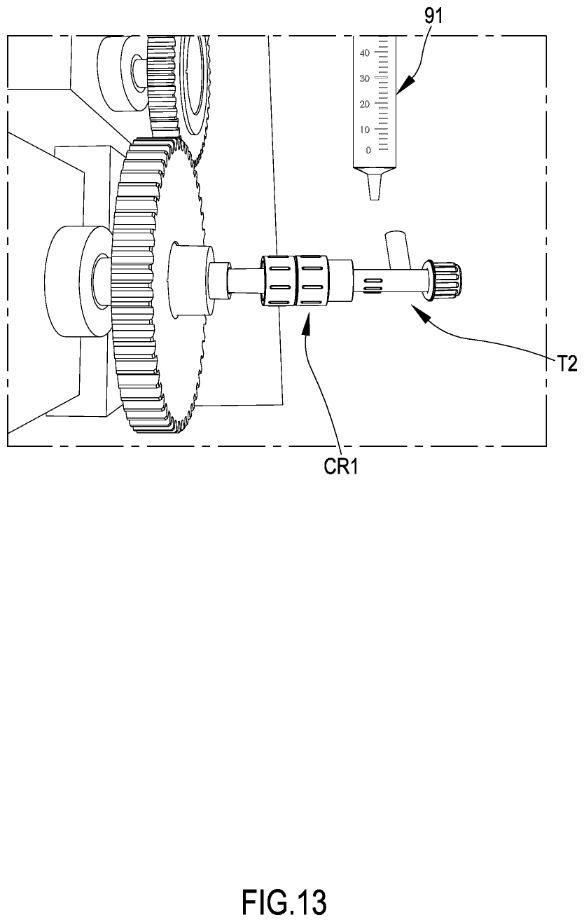

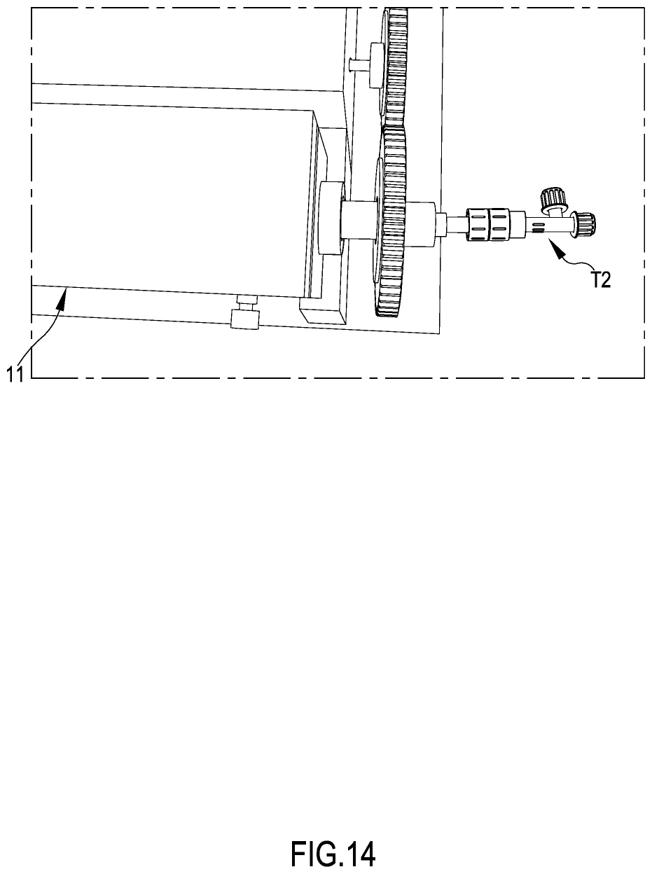

T3 arranged downstream of the bioreactor without possible air bubbles, the T-shaped connector T2 arranged upstream of the bioreactor is closed using a cap (FIG. 11).
1.7 Subsequently, the syringe (91) with the cell suspension residue is rotated by about 90° with respect to the plane on which it lies (FIG. 12); in this position the plunger of the syringe (102) is re-inserted at the open end of the syringe (FIG. 12) by inserting the insulating black part only so as not to create pressure inside the scaffold. Subsequently, the syringe (91) can be unscrewed from the T-shaped connector T2 upstream of the bioreactor without forming air bubbles (FIG. 13) and the end of the connector is closed using a cap (FIG. 14).
1.8 A hot fresh growth medium (as previously defined) is added into the bioreactor chamber until the seeded scaffold present in the bioreactor chamber is half-submerged in the growth medium.
1.9 A continuous rotation is then applied along the longitudinal axis of the scaffold, for example with a rotation speed comprised between 1.5 and 2 rpm, for 24 hours. The rotation allows the uniform cell adhesion on the lumen of the scaffold, allows the scaffold to remain continuously wet by the growth medium present in the bioreactor chamber and it allows the through-flow of the nutrients between the medium present in the chamber (outside the scaffold) and the cell suspension one seeded in the lumen of the scaffold.
1.10 Incubating, preferably for 24 hours at 37° C. with 5% of CO₂, the scaffold housed in the bioreactor chamber (under rotation).

Advantageously, the seeding method created by the Applicant allows to operate under sterility conditions. Furthermore, the present seeding method subject of the present invention is rapid, reproducible, and advantageously allows to prepare a scaffold having the lumen surface (internal) with endothelial cells homogeneously and uniformly adhered along the entire length of the scaffold (from the proximal part to the medial part up to the distal part). The present seeding method subject of the present invention allows to seed the cells eliminating both the air bubbles present in the bioreactor-scaffold system and the air bubbles that are formed, hence avoiding to damage the cells. Basically, each step of the present method is standardised and reproducible and it optimises the cost and the operating time.

### Experimental Evidence of the Seeding Method

To prove the effectiveness of the seeding method subject of the present invention, the following analysis were conducted.

The viability of the cells adhered to the scaffold was assessed using an assay which uses resazurin (trade name Alamar Blue, name IUPAC 7-hydroxy-10-oxidophenoxazin-10-ium-3-one, CAS 550-82-3) as reagent. Such assay consists in a metabolic reaction that allows to quantify cell viability due to the oxidation-reduction of the indicator (resazurin) which is reduced to resofluorine, a pink fluorescent compound in the presence of reducing atmosphere of a vital cell. After 24 hours of adhesion, the seeded scaffold is removed from the grips. Subsequently, the scaffold is sectioned (cutting it) into three areas measuring about 2 cm each depending on the distance from the site of injection of the cell suspension: proximal, medial and distal. Subsequently, each section is divided into 4 parts measuring about 1 cm². 3 samples each representing each region (proximal, medial, distal) of the scaffold with adhered endothelial cells were selected for the assay with resazurin. Each sample is positioned in a well of a 24-well dish and incubated with 1 ml of a 0.02 mg/ml resazurin sodium salt (Sigma Aldrich, R7017) solution with fresh growth medium preferably for 3 hours at 37° C. with 5% of CO₂. The reaction that is developed between the 0.02 mg/ml resazurin sodium salt solution with fresh growth medium and the scaffold sample (with the adhered endothelial cells) is analysed using the A.U. (arbitrary unit of fluorescence) detection at 590 nm by using a spectrofluorometer.

A further analysis conducted is the assessment of the amount of genomic DNA present in the cells adhered on the samples of the scaffold previously used for the assay with resazurin. The genomic DNA is extracted from the adhered cells by a scaffold through lysis and it is subsequently quantified using Quant-iT™ PicoGreen™ dsDNA Assay (P7589, Invitrogen, Molecular Probes) where the fluorescent stain of the nucleic acids (PicoGreen) allows—through a standard reference curve—to determine the concentration of genomic DNA in solution.

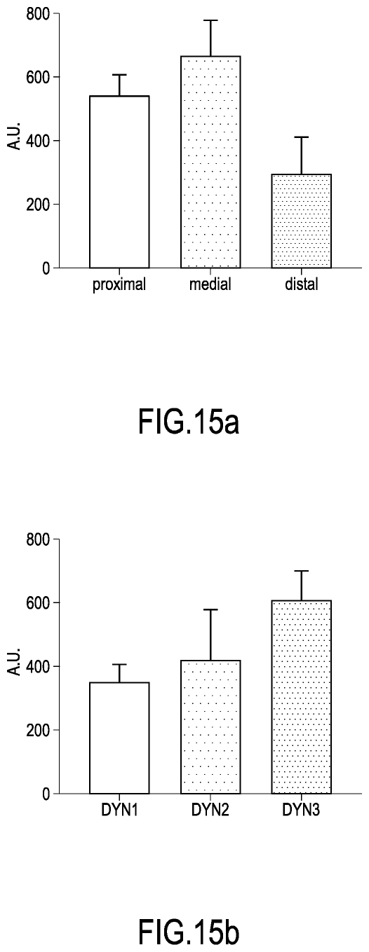

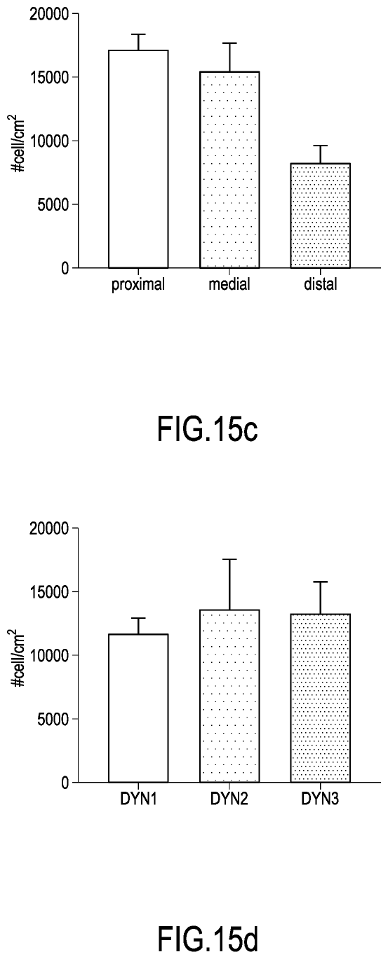

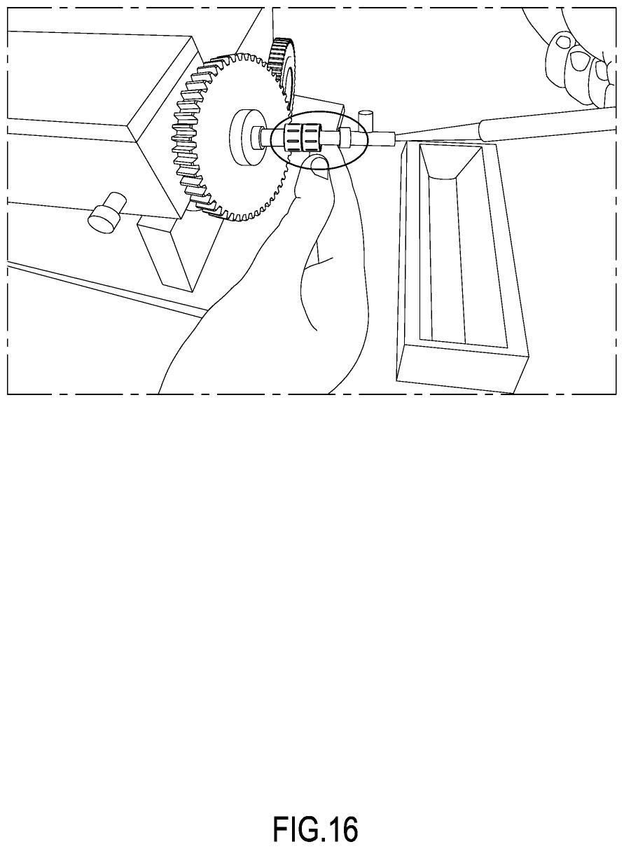

In FIGS. 15A and 15B represented in the chart are the values obtained using the assay with resazurin on samples representing each region (proximal, medial, distal) of a scaffold seeded with endothelial cells and incubated for 24 hours in three different experiments (named DYN1, DYN2 and DYN3). The charts in FIGS. 15A and 15B show a good adhesion and viability of the endothelial cells.

This data was confirmed by the quantification of the genomic DNA (FIGS. 15C and 15D) calculated considering that the genomic DNA content of an endothelial cell is of about 7 pg.

No significant cell viability difference was observed among the various proximal, medial and distal sections of the scaffolds seeded with endothelial cells. In particular, cell viability and the number thereof can be compared along the length (main axis of the scaffold) in the proximal, medial and distal portions thereof. With the aim of supporting this evidence, costaining was conducted using DAPI (4′,6-Diamidino-2-Phenylindole, Dihydrochloride, D1306, ThermoFisher scientific) and Rhodamine-Phalloidin (R415, ThermoFisher scientific) on samples representing each region (proximal, medial, distal) of a scaffold seeded with endothelial cells and incubated for 24 hours. The two DAPI and Rhodamine-Phalloidin reagents are specific respectively for the nuclear detection and for actin filaments (F-actin), morphological components of a live cell, visible after the staining using a fluorescence microscope or a confocal microscopy. After 24 hours of culture, these results show that the endothelial cells are vital and distributed on the lumen of the scaffold in a uniform fashion. In particular, these results show a 90% cell confluence. Furthermore, conducted on samples representing each region (proximal, medial, distal) of a scaffold seeded with endothelial cells and incubated for 24 hours are gene expression analysis for markers typical of endothelial cells: for example, the Von Willebrand factor (VWF), cluster of differentiation 31 (CD31), vascular cell adhesion molecule 1(VCAM-1). In order to conduct a gene expression evaluation, the total RNA is extracted from cells (endothelial in this case) and after reverse transcription at cDNA is quantified using a specific Taqman Gene Expression Assay (ThermoFisher Scientific) using the real-time PCR technique. Functional levels for gene expression of the markers listed previously are indicators of good functionality and viability of the cells adhered on the lumen of the scaffold.

Lastly, H&E “Haematoxylin and Eeosin” staining analysis is conducted on samples of a scaffold seeded with endothelial cells according to the present invention with the aim of evaluating the distribution of the cells and the morphology thereof, and an immunofluorescence assay for specific endothelial functionality markers.

In conclusion, the present seeding method subject of the present invention revealed to be efficient in that it guarantees a homogeneous, uniform and reproducible seeding of vital endothelial cells along the entire lumen.

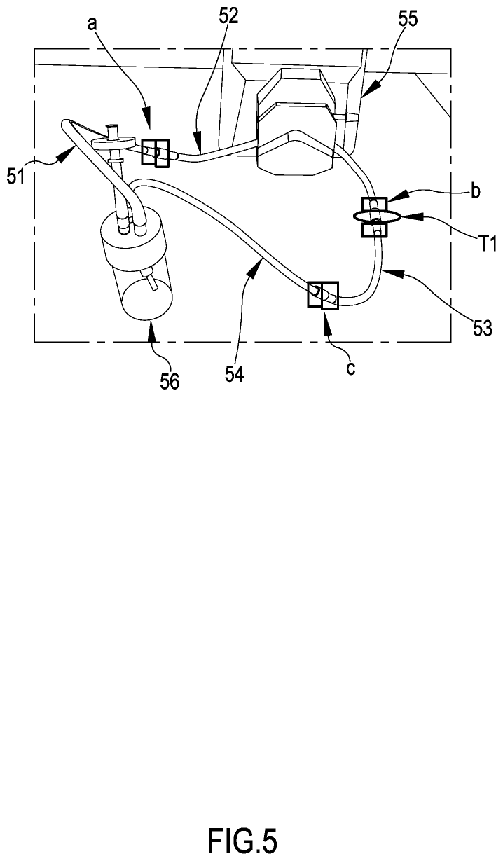

(2) Method for Connecting a Perfusion Circuit to a Bioreactor-Scaffold System (Perfusion Method) according to the First Embodiment (FIGS. 5-8, 18-21).

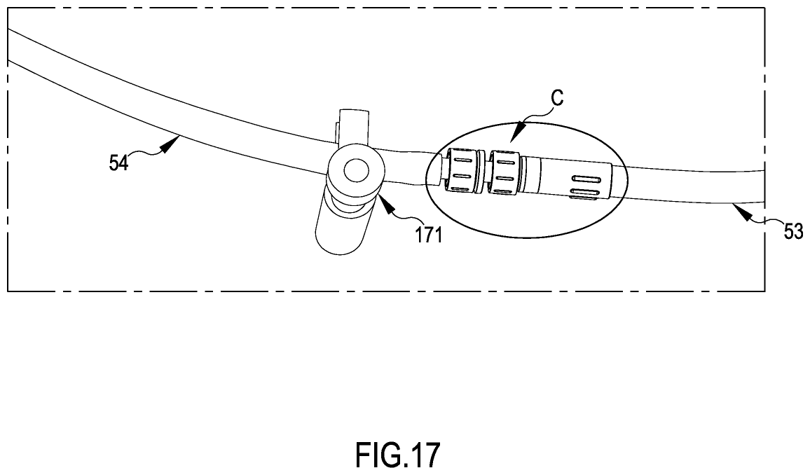

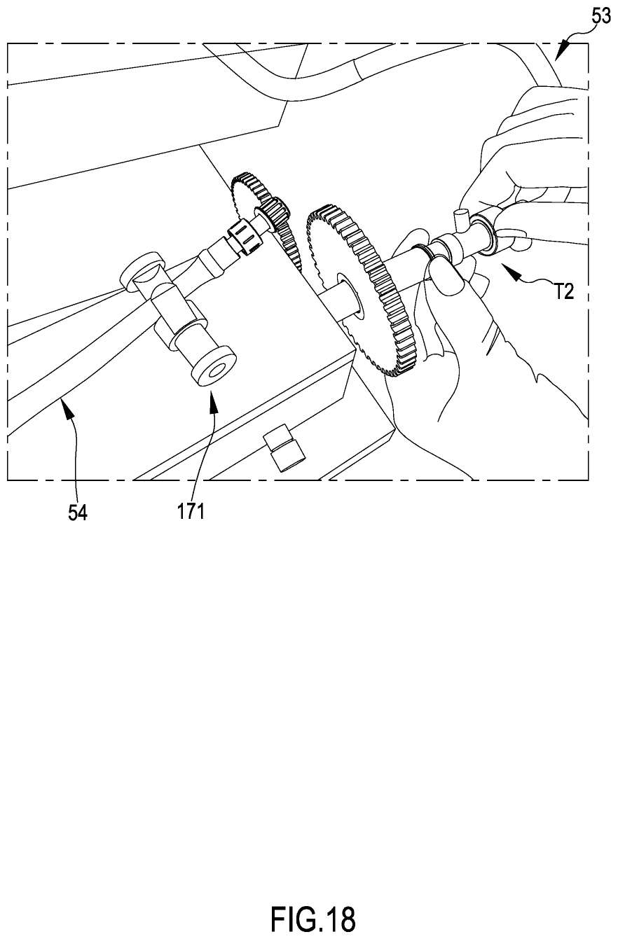

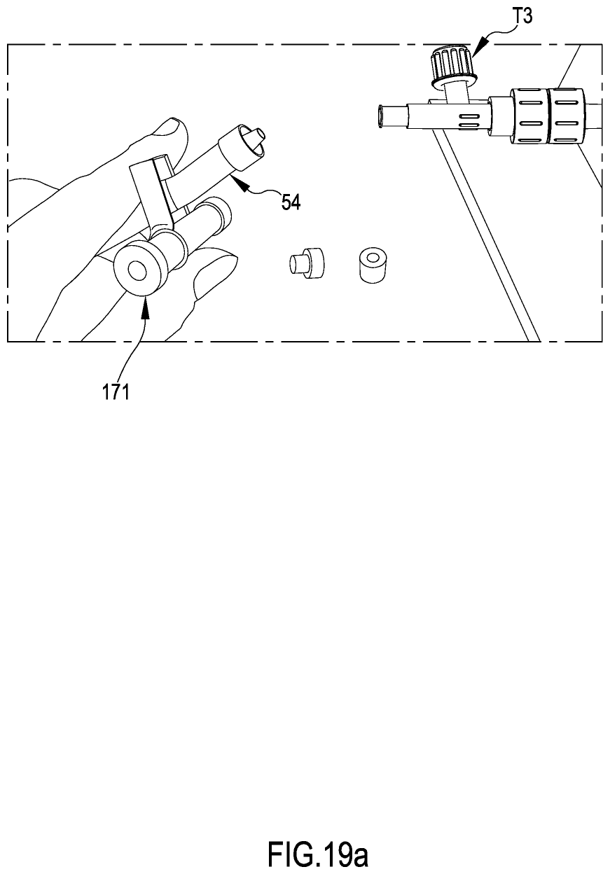

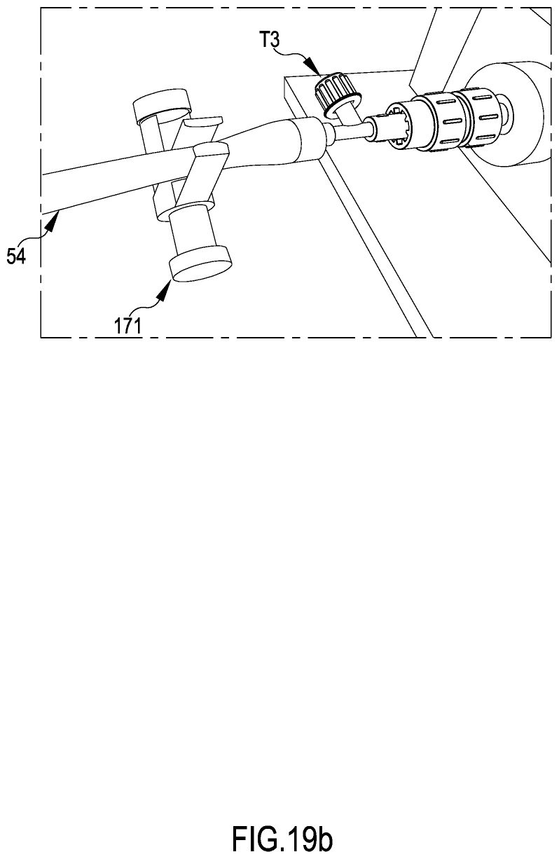

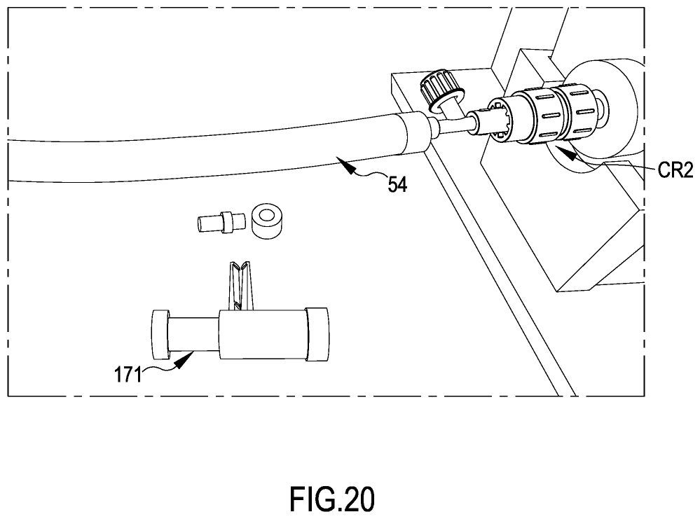

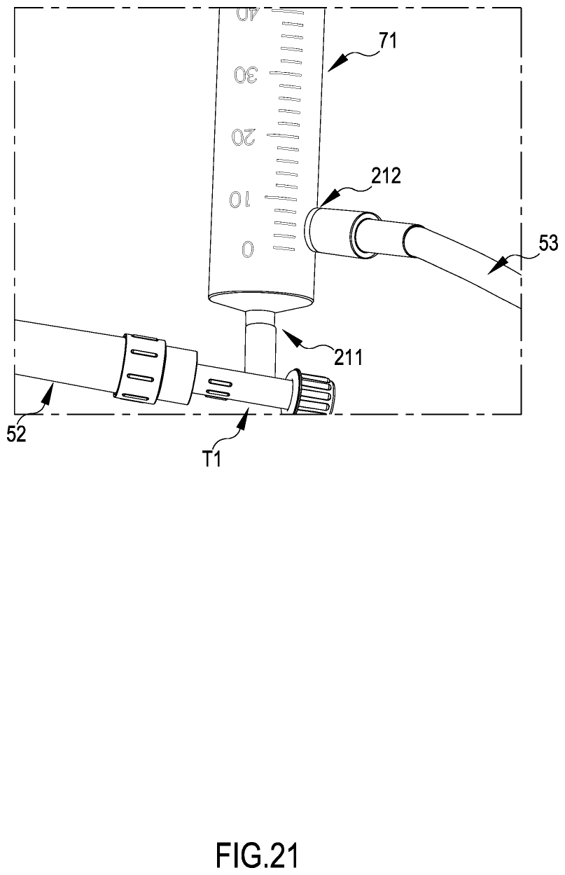

The connection method is based on the following sequential steps, subsequent to seeding (method for seeding a cell culture in the lumen of a scaffold according to the embodiment described above under point (1)) and at 24 hours from adhesion of the endothelial cells:
2.1 Place the tube or under-pump (FIG. 5; 52) of the closed perfusion circuit under the head of the peristaltic pump (FIG. 5; 55), which—upon activation—generates a peristaltic force capable of suctioning fluids, hot fresh growth medium in this case. The closed perfusion circuit is filled due to the suctioning, by the tube (FIG. 5; 51) of the perfusion circuit connected to the reservoir (FIG. 5; 56), of the hot fresh growth medium which is previously poured into the reservoir once closed. Fill the tubes of the perfusion circuit with the hot fresh growth medium until the fresh growth medium returns to the reservoir through the tube 54 (FIG. 5; 54) of the closed perfusion circuit. Place the bioreactor-scaffold system under the same sterility conditions as the perfusion circuit.
2.2 Open the upper and lateral ends of the T-shaped connector T2 arranged upstream of the bioreactor.
2.3 Upon removing the caps from the upper and lateral ends of the T-shaped connector T2 arranged upstream of the bioreactor, the possible creation of air bubbles is compensated by manually adding (preferably using a pasteur pipette) having the same volume as the hot fresh growth medium (FIG. 16).
2.4 Occlude the tube 54 of the closed perfusion circuit, preferably using a clamp (FIG. 17; 171) in a position proximal to the connector between the tube 54 and the tube 53 of the perfusion circuit (FIG. 17). Be careful to keep the head of the pump closed so as to prevent the emptying of the tube 53 of the closed perfusion circuit.
2.5 Keep the tube 53 of the closed perfusion circuit in vertical position, unscrew the connector arranged between the tube 53 and the tube 54 of the perfusion circuit and preferably cap the tube 54 of the perfusion circuit using a cap.
2.6 Screw the tube 53 of the perfusion circuit to the open lateral end of the T-shaped connector T2 upstream of the bioreactor at a lateral access thereof. The connector upstream of the bioreactor must always be kept in vertical position (FIG. 18).
2.7 Open the T-shaped connector T3 downstream of the bioreactor by unscrewing the cap of the lateral opening.
2.8 Remove the cap from the connector of the tube 54 of the perfusion circuit (FIG. 19A) and screw it onto the lateral opening of the T-shaped connector downstream of the bioreactor (FIG. 19B).
2.9 Remove the clamp 171 which occludes the tube 54 of the perfusion circuit (FIG. 20).
2.10 Fill the element for the removal of the air bubbles (FIG. 21, 71), defined with the technical expression of bubble trap in the context of the present invention. The bubble trap consists of an element represented by a chamber closed using a cap and having two accesses serving as an outflow and inflow. The bubble trap chamber contains a volume of liquid (hot fresh growth medium in this specific case) and a volume of air that traps possible air bubbles present in the perfusion liquid which flows through the two accesses of the bubble trap chamber.

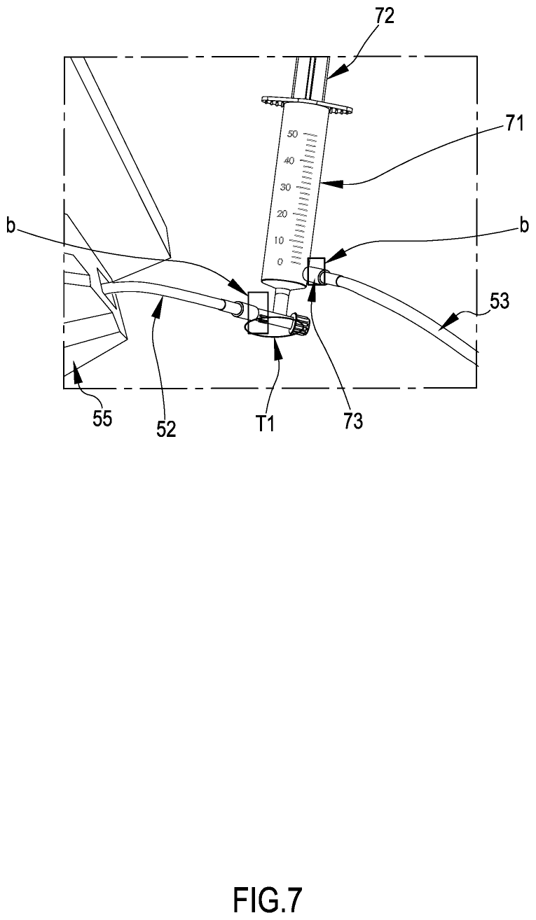

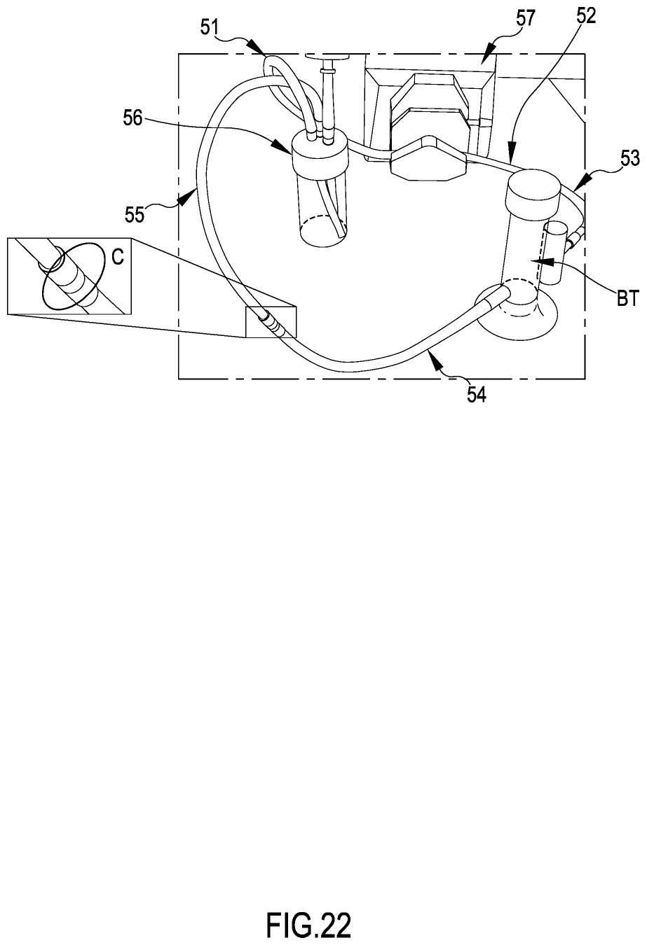

Fill the bubble trap with hot fresh growth medium so as to leave a given air volume and close the chamber as well as its accesses using the respective caps.
2.11 Connect the bubble trap to the perfusion circuit previously connected to the bioreactor-scaffold system as follows (FIG. 7):
a. close the tube 53 of the perfusion circuit using a clamp arranged proximally to the connector which connects the tube 53 to the lateral end of the T-shaped connector T1 (arranged between the tube 53 and the tube 52 of the perfusion circuit) and unscrew it (FIG. 7; 52).
b. preferably, close the tube 53 of the perfusion circuit using a cap. Such operation prevents the emptying of the tube 53 of the perfusion circuit.
c. cap the lateral end of the T-shaped connector T1 (arranged between the tube 53 and the tube 52 of the perfusion circuit) and open the upper end thereof.
d. open the inflow access of the bubble trap chamber and connect it to the access of the upper end and arranged vertically with respect to the T-shaped connector T1.
e. open the outflow access of the bubble trap and connect it to the tube 53 of the perfusion circuit, being careful not to twist the tube at all.
f. keep the bubble trap in vertical position.
g. remove the clamp 171 from the tube 53 of the perfusion circuit just connected to the bubble trap chamber. Start the pump and open the cap of the upper end of the T-shaped connector T2 upstream of the bioreactor so as to eliminate possible air bubbles formed in the tube 53 during the process preventing them from reaching the scaffold. The peristaltic force applied by the pump to the assembled system, consisting of the perfusion circuit and the bioreactor-scaffold system will allow the perfusion of the scaffold.
h. after such verification, close the T-shaped connector T2 upstream of the bioreactor using the cap thereof.
   (3) Method for Connecting a Perfusion Circuit to a Bioreactor-Scaffold System (Perfusion Method) according to a Second Preferred Embodiment (FIGS. 22-27).

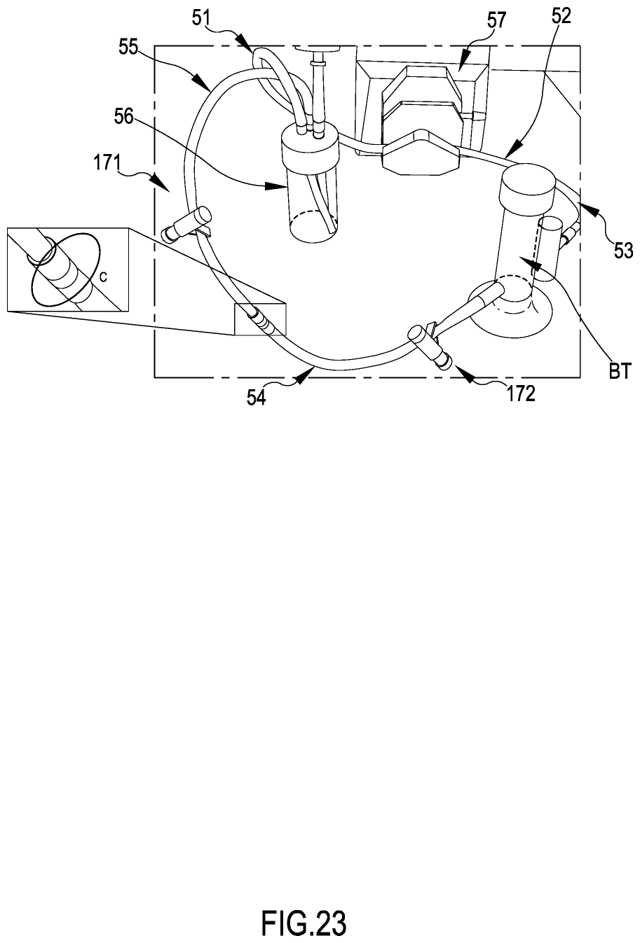

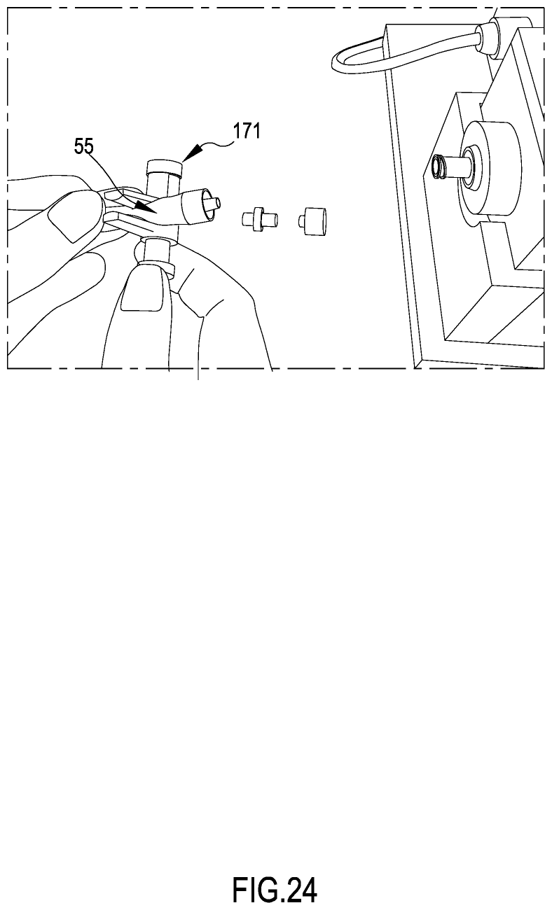

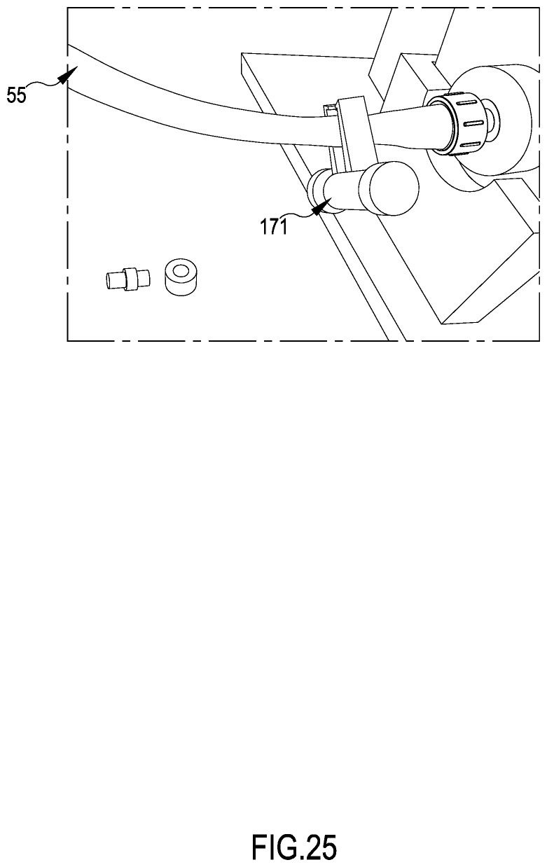

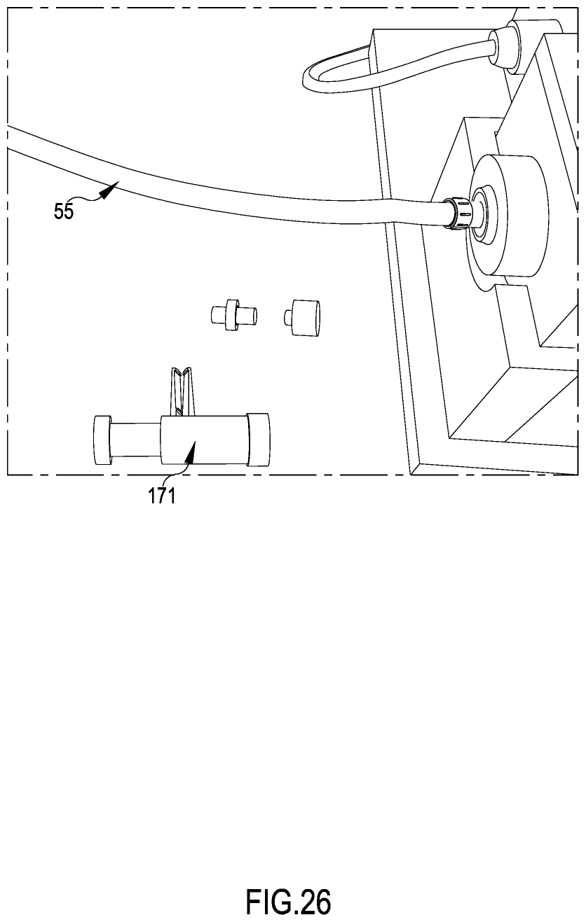

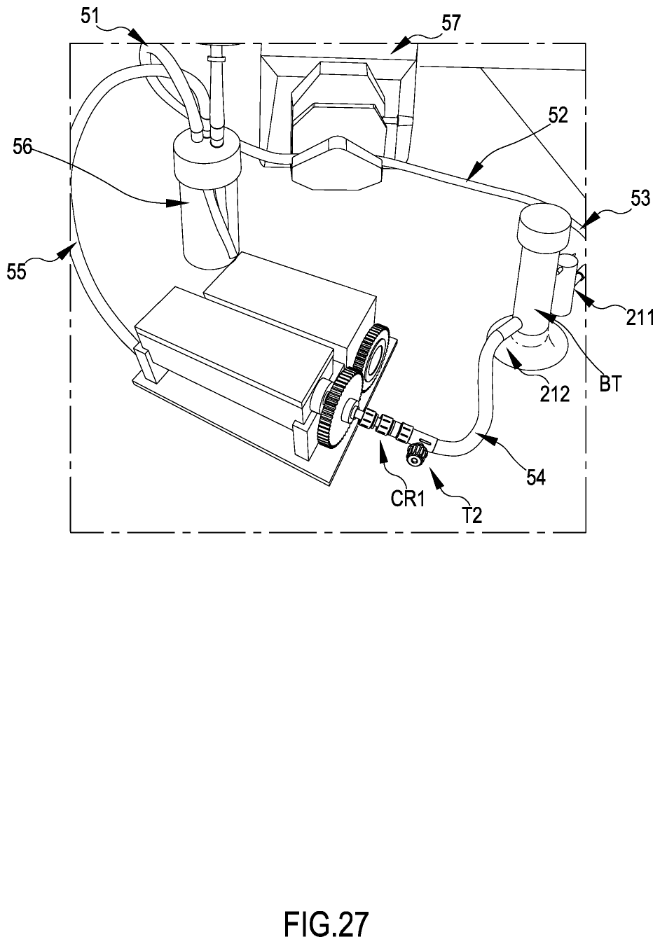

The connection method is based on the following sequential steps, subsequent to seeding (method for seeding a cell culture in the lumen of a scaffold according to the embodiment described above under point (1)) and at 24 hours from adhesion of the endothelial cells:
3.1) Connect—under sterility conditions—the tubes of the perfusion circuit (FIG. 22; 51; 52; 53; 54; 55), the element for removing the air bubbles (FIG. 22; BT), defined in the context of the present invention with the technical expression bubble trap, and the reservoir (FIG. 22; 56). The element for removing the air bubbles or bubble trap (Bt) consists of an element represented by a closed chamber, preferably made of glass, using a cap and having two asymmetric accesses: the access with the tap and connecting nozzle serves as an inflow (FIG. 27, 211) while access with the connecting nozzle only serves as an outflow (FIG. 27, 221). The bubble trap chamber contains a volume of liquid (hot fresh growth medium in this specific case) and a volume of air that traps possible air bubbles present in the perfusion liquid which flows through the two accesses of the bubble trap chamber.
3.2) Place the under-pump (FIG. 22; 52) of the closed perfusion circuit under the head of the peristaltic pump (FIG. 22; 57), which—upon activation—generates a peristaltic force capable of suctioning fluids, hot fresh growth medium in this case. The closed perfusion circuit is filled due to the suctioning, by the tube (FIG. 22; 51) of the perfusion circuit connected to the reservoir (FIG. 22; 56), of the hot fresh growth medium which is previously poured into the reservoir (FIG. 22; 56) in turn closed. Fill all the tubes of the perfusion circuit, the bubble trap (FIG. 22, BT) and the reservoir (FIG. 22; 56) with a liquid perfusion, such as a hot fresh growth medium (as defined above), until the hot fresh growth medium returns to the reservoir through the tube 55 (FIG. 22; 55) of the closed perfusion circuit. The BT and the reservoir are filled so as to leave a given air volume. In particular, the bubble trap (FIG. 22, BT) is filled so that said bubble trap chamber has a first part of the volume thereof filled with said fresh growth medium and a second part of the volume thereof filled with air, said second part of said volume having the function of trapping the air bubbles present in the perfusion liquid (hot fresh growth medium) which flows through said access serving as an inflow (211) and said access serving as an outflow (212) of the bubble trap (BT) (FIG. 22).
3.3) Occlude the tube 54 (FIG. 23), preferably using a clamp (FIG. 23; 172) in a position proximal to the BT and move the tap of the BT to the closing position (in perpendicular position with respect to the tubes of the perfusion circuit).
3.4) Place the bioreactor-scaffold system under the same sterility conditions as the perfusion circuit.
3.5) Occlude the tube 55 (FIG. 23), preferably using a clamp (FIG. 23; 171; FIG. 17; 171) in a position proximal to the connection C with the tube 54 (FIG. 23; C). Open the upper and lateral ends of the T-shaped connector T2 arranged upstream of the bioreactor (FIG. 4; T2).
3.6) Upon removing the caps from the upper and lateral ends of the T-shaped connector T2 (FIG. 4) arranged upstream of the bioreactor, the possible creation of air bubbles is compensated by manually adding (preferably using a pasteur pipette) having the same volume as the hot fresh growth medium (FIG. 16).
3.7) Keep the tube 54 (FIG. 23) of the closed perfusion circuit in vertical position, unscrew the connector arranged between the tube 54 (FIG. 23) and the tube 55 (FIG. 23) of the perfusion circuit and preferably cap the tube 55 of the perfusion circuit using a cap (FIG. 24).
3.8) Screw the tube 54 (FIG. 27) of the perfusion circuit to the open lateral end of the T-shaped connector T2 (FIG. 27) upstream of the bioreactor at a lateral access thereof. The connector upstream of the bioreactor must always be kept in vertical position (FIG. 18 or 27).
3.9) Open the T-shaped connector T3 (FIG. 4) downstream of the bioreactor by unscrewing the cap of the lateral opening.
3.10) Unscrew the rotary connector CR2 together with the T-shaped connector T3 (FIG. 4), holding the toothed wheel R (FIG. 4) still and screw the connector of the tube 55 (FIG. 25) on the lateral opening of the scaffold-holder 14a (FIG. 1) (FIG. 25).
3.11) Remove the clamp 171 which occludes the tube 55 of the perfusion circuit (FIG. 26).
3.12) Position the bioreactor-seeded scaffold system connected to the perfusion circuit at about 37° C. and at about 5% of CO₂, place the under-pump 52 (FIG. 27) in free position, open the tap of the bubble trap BT (FIG. 27) by positioning it parallel to the tubes of the perfusion circuit, remove the clamp 172 (FIG. 27) and then start the pump (FIG. 27, 57). The peristaltic force applied by the pump to the assembled system, consisting of the perfusion circuit and the bioreactor-scaffold system will allow the perfusion of the scaffold.

Advantageously, the connection method (perfusion method) described herein, both in the first embodiment and in the second embodiment described above, allows to connect a perfusion circuit of a scaffold, preferably tubular, to the bioreactor-seeded scaffold system. This procedure allows to prevent the formation of air bubbles and prevents the bubbles, should they be formed, from reaching the scaffold seeded with endothelial cells of the bioreactor-scaffold system. Furthermore, the air bubbles possibly already present in the perfusion circuit do not reach the scaffold due to the presence of the bubble trap (FIG. 21, 71; FIG. 27, BT) in the circuit. In this manner, the method created by the Applicant is capable of ensuring complete absence of air bubbles in the lumen of the scaffold. This system meets the requirements set forth by the configuration of the bioreactor. All details indicated and described are required to make the method independent from the operator and thus for ensuring the reproducibility of the results during the in vitro generation of a construct with functional endothelium at industrial level. In addition, this method allows to be able to operate under sterility conditions, being based on simple actions. Furthermore, the quick and traceable procedure allows to reduce the risk that the air bubbles come into contact with the cells, thus avoiding damaging the endothelial cells adhered on the lumen of the scaffold, preferably tubular. Said perfusion method allows to obtain engineered vascular constructs/tissues having a scaffold having a lumen covered with a continuous (i.e. having a monolayer of confluent cells) and functional endothelium.

### Experimental Evidence of the Connection Method

The experimental analysis regarding the evaluation of the method for connecting the perfusion circuit to the bioreactor-scaffold system are the same ones applied for the evaluation of the seeding method of a cell culture in a scaffold preferably tubular.

In the context of the present invention the perfusion circuit (FIG. 5 and FIG. 22) is defined as an assembly of: tubes (FIG. 5; 51-54 or FIG. 22; 51-55), a reservoir (FIG. 5 or FIG. 22; 56), a peristaltic pump (FIG. 5; 55 or FIG. 22; 57) and an element for removing the air bubbles (FIG. 21, 71 or FIG. 22, BT). Said element for removing the air bubbles can be present in the perfusion circuit prior to the connection of the perfusion circuit to the bioreactor-seeded scaffold system (second embodiment of the perfusion method) or, alternatively, it may be inserted into the perfusion circuit after the connection of the perfusion circuit to the bioreactor-seeded scaffold system (first embodiment of the perfusion method). Said tubes are made of biocompatible material and are connected to each other so as to allow the perfusion of the scaffold (FIG. 21 and FIG. 27), preferably tubular, housed in the bioreactor 11, by means of the peristaltic pump (FIG. 5; 55, FIG. 22; 57) (in such case, Masterflex®, L/S Digital Dispensing Pump Drives 07551-20, Cole-Parmer) with the Easy-Load II 77200-62 (Masterflex, Cole-Parmer) head. With reference to then second embodiment of the perfusion method described above and illustrated in FIG. 27, the perfusion circuit mainly consists of five tubes with an inner diameter of 3/16″: a first tube 51 for suctioning from the reservoir, a second under-pump tube 52, a third tube 53 which connects the circuit to the bubble trap BT, a fourth tube 54 which connects the BT to the T-shaped connector T2 upstream of the bioreactor-scaffold system, a fifth tube 55 for return to the reservoir 56 connected to the T-shaped connector T3 downstream of the bioreactor-scaffold system.

With reference to the first embodiment of the perfusion method described above and represented in FIG. 5, the tube 51 is connected to the under-pump tube 52, the under-pump tube 52 to the BT, the BT to the tube 53, the tube 53 to the T-shaped connector T2 upstream of the bioreactor-scaffold system, the tube 54 to the T-shaped connector T3 downstream of the bioreactor-scaffold system and to the reservoir 56.

The reservoir (FIG. 5 or FIG. 22, 56) is the element containing the hot fresh growth medium (for example Endothelial Growth Medium EGM, Sigma Aldrich) from which the tube 51 (FIG. 5 or FIG. 22) suctions and to which the tube 54 (FIG. 5) or 55 (FIG. 22) returns, keeping the entire bioreactor-seeded scaffold system in a closed system. The reservoir (FIG. 5 or FIG. 22, 56) is under atmospheric pressure, due to a 0.22 μm filter present in the cap of the reservoir, which guarantees the sterility of the air.

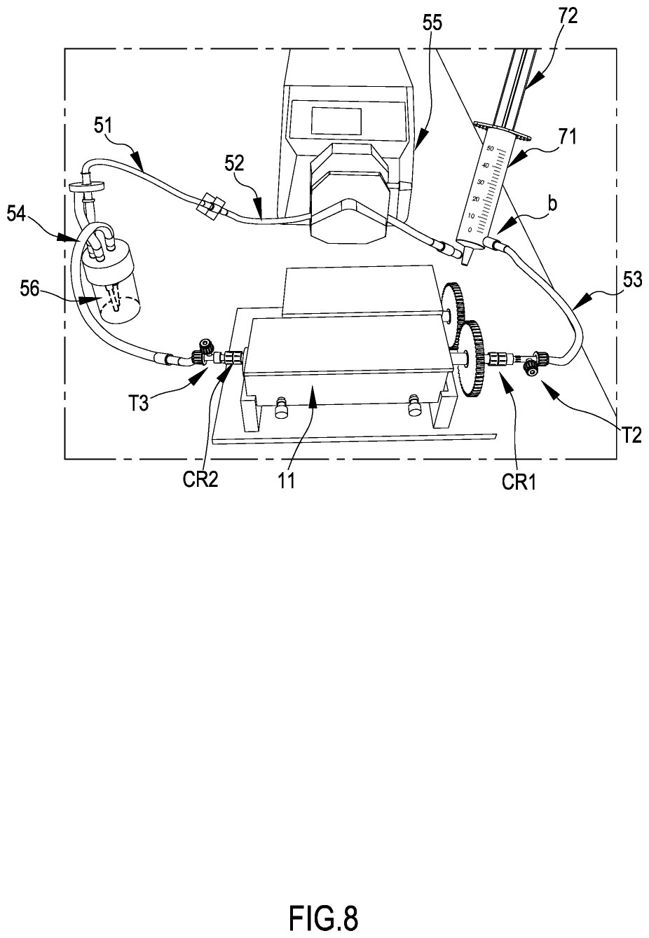

Also forming an object of the present invention are the following preferred embodiments RPn, as indicated below.
RP1. A process for producing vascular tissues, preferably scaffold (FIG. 2; 21), for testing medicinal products, said process comprises applying:
   a method for seeding an endothelial cell culture in the lumen of a scaffold (FIG. 2; 21) to obtain a seeded scaffold; said seeded scaffold being present in a bioreactor (FIG. 4; 11), to obtain a bioreactor-seeded scaffold system and, subsequently,
   a method for perfusion of the endothelial cells present in the lumen of said seeded scaffold; said perfusion method being obtained by connecting a perfusion circuit (FIGS. 6; 51, 52, 53, 54, 55, and 56) to said bioreactor-seeded scaffold system;
RP2. The process according to claim RP1, wherein said method for seeding an endothelial cell culture in the lumen of a scaffold comprises:
   mounting a scaffold (21), preferably an electrospun silk fibroin tubular scaffold, on the grips of a scaffold-holder (FIG. 1; 13, 13a, 13b) and housing said scaffold-holder (13, 13a, 13b) with the scaffold (21) in the bioreactor chamber (11), to obtain a bioreactor-scaffold system (FIG. 4; 11, 21);
   injecting a fresh growth medium into the lumen of said scaffold (21) fixed on said scaffold-holder (13) arranged inside the bioreactor chamber (11);
   adding said fresh growth medium into the bioreactor chamber (11) where said scaffold-holder (13) with the scaffold (21) is present already injected with said growth medium;
   leaving, preferably for a time interval comprised between 1 hour and 18 hours at a temperature comprised between 20° C. and 30° C., preferably 25° C., said growth medium in the lumen of the scaffold (21) and in the bioreactor chamber (11) where said scaffold-holder (13) with the scaffold (21) is present already injected with said growth medium;
   clearing the internal of the lumen of the scaffold (21) and of the bioreactor chamber of the culture medium;
   releasing a cell suspension consisting of said fresh growth medium and endothelial cells of the HUVECs type into a container of the syringe type (FIG. 10; 91) mounted on the connector element (T2) arranged upstream of the bioreactor (11) by means of the element (CR1); said cell suspension is released into the lumen of the scaffold (21) present in the bioreactor chamber (11) with a continuous flow so that the flow speed allows said cell suspension to drip into the container (T2) without generating the air bubbles and pushing any air bubbles present in the lumen of the scaffold (21), towards the opening of the connector (T3) arranged downstream of the bioreactor (11) allowing the outflow thereof;
   adding said fresh growth medium into the bioreactor chamber (11) where said scaffold-holder (13) with the scaffold (21) is present containing said cell suspension therein;
   incubating, preferably for 24 hours at 37° C. in presence of 5% of CO₂, the scaffold (21) housed in the bioreactor chamber (11).
RP3. The process according to RP1 or RP2, wherein said method for the perfusion of the endothelial cells present in the lumen of said seeded scaffold comprises:
   preparing a closed perfusion circuit (FIG. 5) comprising the tubes (FIGS. 5; 51, 52, 53, 54, 55 and 56);
   occluding the tube (54) of the perfusion circuit using a closing element of the clamp type (FIG. 17; 171) in a position proximal to the connector (C);
   unscrewing the connector (C) arranged between the tube 53 and the tube 54 (FIG. 5; FIG. 17) of the perfusion circuit;
   screwing the tube (53) of the perfusion circuit to the open lateral end of the connector (T2) upstream of the bioreactor (FIG. 18) at a lateral access thereof;
   opening the connector (T3) downstream of the bioreactor and unscrewing the cap of the lateral opening (FIG. 19a);
   connecting the tube (54) of the perfusion circuit to the lateral opening of the connector (T3) arranged downstream of the bioreactor (FIG. 19b) and removing the closing element of the clamp type (FIG. 20; 171).
RP4. The process according to RP3, wherein an element (71) represented by chamber closed using a cap (72) and having two accesses serving as an inflow (211) and as an outflow (212) of the bubble-trap type (FIG. 21) is inserted into the tube (53) of the perfusion circuit (FIG. 8; FIG. 21); said chamber has a volume where a first part thereof is filled with a hot fresh growth medium and where a second part thereof is filled with air so as to trap—in the latter second part of volume—possible air bubbles present in the perfusion liquid which flows through said accesses (211 and 212).
RP5. The process according to any one of RP1-4, wherein the scaffold, preferably a tubular scaffold, is selected from among polymeric scaffolds of synthetic or natural origin formed by only one polymer or by copolymers, such as for example electrospun silk fibroin or copolymers of polyglycolic acid/polylactic acid (PGA/PLA) or polyglycolic acid/polycaprolactone (PGA/PCL).
RP6. The process according to any one of RP1-5, wherein the endothelial cells are selected from among the cells that form an endothelium of a vascular tissue, such as for example HAOECs (human aortic endothelial cells), HCAECs (human coronary artery endothelial cells), HMEVECs (human dermal microvascular endothelial cells) or HUVECs (human umbilical vein endothelial cells).
RP7. The process according to any one of RP1-6, wherein the growth medium used is the Endothelial Growth Medium (EGM, Sigma Aldrich, 211-500), preferably heated to 37° C.
RP8. A scaffold having a lumen covered with a continuous and functional endothelium obtained by means of the process according to any one of RP1-7.
RP9. Use of the scaffold according to RP8, to conduct in vitro preclinical and clinical tests of a medicinal product to be used in the cardiovascular and peripheral vascular region, such as for example, heart valves, stents, grafts, catheters.

The first phase to be carried out and optimised is the cell seeding phase, preferably endothelial cells, followed by a second critical phase of connecting the perfusion circuit to the system comprising the bioreactor and the scaffold, so as to ensure reliability, effectiveness and reproducibility to the industrialisation process for the in vitro generation of a continuous and functional endothelium.

The success of the industrialisation process subject of the present invention for the production of the engineered vascular tissue/construct, preferably a scaffold having a lumen covered with a functional and continuous endothelium having a confluent cell monolayer, mainly consists in the success relating to the phase of seeding and connecting the perfusion circuit to the bioreactor-scaffold system, so as to globally guarantee the elimination of air bubbles in a reliable and reproducible manner. The seeding method depends on the cell source and on the density thereof, on the chemical and porosity properties and on the full removal of the air bubbles from the lumen of the scaffold during the injection of the cell suspension. On the other hand, the method for connection between the perfusion circuit and the bioreactor-scaffold system is based on maintaining the sterility of the assembled system and on the guarantee of absence of air bubbles that can come into contact with the seeded scaffold. It should be observed that the formation of air bubbles must be avoided given that the air bubbles can damage the cells, jeopardising the viability thereof with ensuing lack of endothelisation of the scaffolds.

The description of the present invention shows that the choice of the method for connecting the perfusion circuit to the bioreactor-scaffold system depends on the method for seeding the previously optimised endothelial cells due to the fact that said connection method must be suitable to the experimental setup and to the perfusion needs and the position chosen for this system in the incubator.

The process for seeding a scaffold, preferably a tubular scaffold, is one of the factors crucial towards in vitro generation of functional engineered vascular constructs, using confluent endothelium, as shown in the description of the present invention. This process is responsible for a uniform and homogeneous distribution of endothelial cells in the lumen same case applying to the adhesion of the cells to the surface. The description of the present invention shows that the choice of the most appropriate seeding method, adapting it to each bioreactor-scaffold system, defines the reproducibility of the process, showing the advantages thereof in the reproducibility of the results.

Also in preclinical tests, as well as others, there arises the need for creating reproducible methods for the large-scale production of functional vascular constructs.

Thus, the seeding method of the present invention, well defined and traceable, guarantees a highly uniform distribution in terms of adhesion of endothelial cells and good reproducibility of the results, required for a laboratory whose activity focuses on the production of in vitro vascular constructs as test models of the preclinical field as well as other fields.

After 24 hours of adhesion, the cells adhered in the lumen of the scaffold, mainly tubular, must maintain their morphology and viability so as to obtain a homogeneous vascular endothelium. Thus, it is important to avoid any cell alteration during the connection process which could alter the state of cell adhesion, with possible loss of the vascular layer subject of growth (being formed).

The known methods for connection between the bioreactor and the perfusion circuit cause cell suffering with ensuing detachment—even partial—of the endothelial cells from the luminal surface, so that the formation of a functional endothelial layer is slowed or hindered. Furthermore, the known methods for connection between the bioreactor and the perfusion circuit do not guarantee the absence of air bubbles, that may be formed due to possible torsions or compressions (full or partial) of the connection tubes during the perfusion. It is important to absolutely prevent the contact between said air bubbles and the seeded scaffold which is avoided by introducing an element, for example a bubble-trap capable of eliminating the air bubbles before they reach the seeded scaffold. This drawback was successfully overcome thanks to the process subject of the present invention which allows to obtain an inner surface of the scaffold covered with a uniform and functional layer of endothelial cells, in particular a confluent cell layer).

Preferred embodiments En of the present invention are described below:
E1. An in-vitro model of a substantially tubular-shaped vascular structure having dysfunctional anatomical and physiological characteristics simulating the same vascular structure of a healthy subject whose vascular structure has been damaged or deformed or deteriorated due to a damage selected from among the group comprising or, alternatively, consisting of aneurysm, stenosis, sclerosis plaques, forms of tumours or cardiomyopathies;
wherein said model comprises or, alternatively, consists of one or more biocompatible porous polymeric supports (scaffolds) capable of promoting a cell adhesion and growth, wherein said scaffold is seeded with endothelial cells which cover a lumen of the scaffold and constitute an endothelium having a single layer of confluent cells, said scaffold being provided with deformities or defects on a tubular structure thereof.
E2. The in vitro model according to E1, wherein said vascular structure is selected from among blood vessels or blood ducts or central or peripheral circulatory system valves; preferably arteries, veins, capillaries, aortic or mitral valve.
E3. The in vitro model according to E1 or E2, wherein said vascular structure is a synthetic vascular structure, and wherein said scaffold consists of electrospun silk fibroin, copolymers of polyglycolic acid/polylactic acid (PGA/PLA) or copolymers of polyglycolic acid/polycaprolactone (PGA/PCL).
E4. The in vitro according to any one of E1-E3, wherein said deformities or said defects of the tubular structure comprise bifurcations, curvatures, elbows, constrictions, dilatations or combinations thereof.
E5. A method for testing a medical device or a drug so as to verify the effectiveness and safety thereof before the in-vivo use thereof on the man or animal, said method comprising the following steps:
   preparing a substantially tubular-shaped scaffold having the dysfunctional anatomical and physiological characteristics suitable to simulate a damage or a deformation or a deterioration due to an aneurysm, stenosis, sclerosis plaques, forms of tumours or cardiomyopathies;
   seeding at least one part of the interior lumen of said scaffold with endothelial cell lines so as to obtain a continuous and homogeneous layer of seeded endothelial cells (seeding method), optionally seeding at least one part of the outer surface of said scaffold with muscle cell lines;
   promoting the growth of said endothelial cells, and optionally said muscle cells, up to obtaining a continuous and uniform layer of endothelial cells, to obtain said in vitro model;
   introducing into said in vitro model a medical device or a drug subject of test, and
   allowing the circulation (perfusion model) in said in-vitro model comprising said medical device or drug of a human whole blood sample, artificial blood or derivatives thereof so as to evaluate the behaviour and the interaction of said medical device or drug with said human whole blood sample, artificial blood or derivatives thereof.
E6. A method or process for the production of an engineered vascular tissue or construct, preferably a scaffold (21) having a lumen covered with functional and continuous endothelium having a confluent cell monolayer, for testing medical or veterinarian products, said process comprising applying:
   a method for seeding an endothelial cell culture in the lumen of a scaffold (21) to obtain a seeded scaffold (21); said seeded scaffold (21) being present in a bioreactor (11), to obtain a bioreactor (11)-seeded scaffold (21) system;
      wherein said seeding method comprises the steps of:
   releasing said endothelial cell culture in form of a cell suspension comprising a fresh growth medium and endothelial cells in a container (91) mounted on a T-shaped connector (T2) arranged upstream of the bioreactor (11) by means of a rotary connector (CR1); followed by
   releasing said endothelial cell culture in the lumen of the scaffold (21) present in the bioreactor chamber (11) with a continuous flow such that the flow speed allows said cell suspension to drip into the T-shaped connector (T2) without generating air bubbles and pushing the air bubbles present in the lumen of the scaffold (21) towards an opening of a T-shaped connector (T3) arranged downstream of the bioreactor (11) allowing the outflow thereof;
      and, subsequently,
   a method for perfusion—with a fresh growth medium having a temperature comprised in the range between 30° C. and 45° C., preferably at 37° C.—of the endothelial cells present in the lumen of said seeded scaffold (21); said perfusion method being obtained by connecting a perfusion circuit (51-56) or (51-57 and BT) to said bioreactor (11)-seeded scaffold (21) system;
      wherein said perfusion method comprises a step of
   partly filling an element for removing the air bubbles (71) or (BT) present in the perfusion circuit with said fresh growth medium, wherein said element for removing the air bubbles (71) or (BT) comprises a chamber, a cap that closes said chamber, an access with inflow function (211) and an access with outflow function (212), wherein said chamber of the element for removing the air bubbles (71 or BT) has a volume and wherein a first part of said volume is filled with said fresh growth medium and wherein a second part of said volume is filled with air, said second part of said volume having the function of trapping the air bubbles present in said fresh growth medium which flows through said access with inflow function (211) and said access with outflow function (212).
E7. The process according to E5 or E6, wherein said method for seeding said endothelial cell culture in the lumen of said scaffold (21) comprises:
   mounting the scaffold (21), preferably an electrospun silk fibroin tubular scaffold, on the grips of a scaffold-holder (13, 13a, 13b) and housing said scaffold-holder (13, 13a, 13b) with the scaffold (21) in the bioreactor chamber (11), to obtain a bioreactor(11)-scaffold (21) system;
      followed by
   injecting the fresh growth medium into the lumen of said scaffold (21) fixed on said scaffold-holder (13) arranged inside the bioreactor chamber (11); followed by
   adding said fresh growth medium into the bioreactor chamber (11) where said scaffold-holder (13, 13a, 13b) with the scaffold (21) is present injected with said growth medium; followed by
   leaving for a time interval comprised between 1 hour and 18 hours at a temperature comprised between 20° C. and 30° C., preferably 25° C., said growth medium in the lumen of the scaffold (21) and in the bioreactor chamber (11) where said scaffold-holder (13) with the scaffold (21) is present injected with said growth medium; followed by
   clearing the internal of the lumen of the scaffold (21) and of the bioreactor chamber (11) of the growth medium; followed by
   releasing said endothelial cell culture in said container (91) according to claim 1, preferably said container (91) is a syringe; followed by
   releasing said cell suspension in the lumen of the scaffold (21) according to claim 1; followed by
   adding said fresh growth medium in the bioreactor chamber (11) where said scaffold-holder (13) with the scaffold (21) is present seeded containing said cell suspension in the lumen; and followed by
   incubating, preferably for 24 hours at 37° C. in presence of 5% of CO₂, the scaffold (21) housed in the bioreactor chamber (11).
E8. The process according to any one of E5-E7, wherein said method for the perfusion of the endothelial cells present in the lumen of said seeded scaffold (21) comprises:
   preparing said closed perfusion circuit comprising the tubes (51), (52), (53), (54), and, optionally, (55);
   occluding the tube (54) or (55) of the perfusion circuit using a closing element (171) in a position proximal to a connector (C), preferably said closing element is a clamp or the like; followed by
   unscrewing the connector (C) arranged between the tube (53) or (54) and the tube (54) or (55) respectively in the perfusion circuit;
   screwing the tube (53) or (54) of the perfusion circuit to an open lateral end of the T-shaped connector (T2) upstream of the bioreactor (11) at a lateral access thereof; followed by
   opening the T-shaped connector (T3) downstream of the bioreactor (11) and unscrewing a cap of a lateral opening of the T-shaped connector (T3); followed by
   connecting the tube (54) or (55) of the perfusion circuit to the lateral opening of the T-shaped connector (T3) arranged downstream of the bioreactor (11) and removing the closing element (171);
      followed, if need be, by
   inserting—between the tube (53) and the under-pump tube (52) of the perfusion circuit—the element for removing the air bubbles (71).
E9. The process according to any one of E6-E8, wherein the element for removing the air bubbles (71) or (BT) is a bubble-trap or the like.
E10. The process according to any one of E5-E9, wherein the scaffold (21), preferably a tubular scaffold, is selected from among polymeric scaffolds of synthetic or natural origin, wherein said polymeric scaffolds are formed by only one polymer or by copolymers, preferably electrospun silk fibroin or copolymers of polyglycolic acid/polylactic acid (PGA/PLA) or copolymers of polyglycolic acid/polycaprolactone (PGA/PCL).
E11. The process according to any of E5-E10, wherein the endothelial cells are selected from among the cells that form an endothelium of a vascular tissue, preferably HAOECs (human aortic endothelial cells), HCAECs (human coronary artery endothelial cells), HMEVECs (human dermal microvascular endothelial cells) or HUVECs (human umbilical vein endothelial cells).
E12. The process according to any one of E6-E11, wherein the growth medium used is the Endothelial Growth Medium comprising fetal bovine serum (2%), adenine (0.2 μg/ml), ammonium metavanadate (0.0006 μg/ml), amphotericin B (0.3 μg/ml), calcium chloride 2H₂O (300 μg/ml), choline chloride (20 μg/ml), copper sulphate 5H₂O (0.002 μg/ml), trioptic acid DL-6,8(0.003 μg/ml), folinic acid (calcium) (0.6 μg/ml), heparin (4 μg/ml), hydrocortisone (2 μg/ml), L-aspartic acid (15 μg/ml), L-cysteine (30 μg/ml), L-tyrosine (20 μg/ml), manganese sulphate monohydrate (0.0002 μg/ml), ammonium molybdate 4H₂O (0.004 μg/ml), nicotinamide (8 μg/ml), nickel chloride 6H₂O (0.0001 μg/ml), penicillin (60 μg/ml), phenol red sodium salt (15 μg/ml), potassium chloride (300 μg/ml), putrescine dihydrochloride (0.0002 μg/ml), pyridoxine hydrochloride (3 μg/ml), sodium metasilicate 9H₂O (3 μg/ml), sodium sulphate 7H₂O (200 μg/ml), sodium selenite (0.01 μg/ml), streptomycin sulphate (100 μg/ml), thiamine hydrochloride (4 μg/ml) and zinc sulphate 7H₂O (0.0003 μg/ml), preferably heated to 37° C.
E13. A scaffold (21) having a lumen coated with a functional and continuous endothelium (21) having a confluent cell monolayer obtained by means of a process comprising the following steps:
   preparing a substantially tubular-shaped scaffold having the dysfunctional anatomical and physiological characteristics suitable to simulate a damage or a deformation or a deterioration due to an aneurysm, stenosis, sclerosis plaques, forms of tumours or cardiomyopathies;
   seeding at least one part of the interior lumen of said scaffold with endothelial cell lines so as to obtain a continuous and homogeneous layer of seeded endothelial cells (seeding method), optionally seeding at least one part of the outer surface of said scaffold with muscle cell lines;
   promoting the growth of said endothelial cells, and optionally said muscle cells, up to obtaining a continuous and uniform layer of endothelial cells, to obtain said in vitro model; preferably wherein said scaffold (21) can be used in an in vitro model according to one from E1-E4; more preferably wherein said process comprises the characteristics of any one from E6-E12.
E14. Use of the scaffold (21) according to any one of E1-E4 or E13, for conducting in vitro preclinical or clinical tests of a medicinal product for human use or of a veterinarian product for animal use to be used in the cardiovascular and peripheral vascular region, preferably valves, heart valves, stents, grafts, catheters, bandages or nets.

### LIST OF REFERENCE NUMBERS

11 bioreactor
13 scaffold-holder
13a scaffold-holder grip
13b scaffold-holder grip
14a lateral opening of the scaffold-holder
21 scaffold
41 inflow of the bioreactor chamber
42 outflow of the bioreactor chamber
51 tube
52 tube or under-pump
53 tube
54 tube
55 head of the peristaltic pump
56 reservoir
57 pump
71 element for removing air bubbles (or bubble trap)
72 cap
91 container, preferably syringe
101 pipette
102 syringe plunger
171 clamp
172 clamp
211 access with inflow function
212 access with outflow function
BT bubble trap
CR1 rotary connector
CR2 rotary connector
T1 T-shaped connector
T2 T-shaped connector
T3 T-shaped connector

1. An in-vitro model of a substantially tubular-shaped vascular structure having dysfunctional anatomical and physiological characteristics simulating the same vascular structure of a healthy subject whose vascular structure has been damaged or deformed or deteriorated due to a damage selected from among the group comprising or, alternatively, consisting of aneurysm, stenosis, sclerosis plaques, forms of tumours or cardiomyopathies;
wherein said model comprises or, alternatively, consists of one or more biocompatible porous polymeric supports (“scaffold”) capable of promoting a cell adhesion and growth, wherein said scaffold is seeded with endothelial cells which cover a lumen of the scaffold and constitute an endothelium having a single layer of confluent cells, said scaffold being made with deformities or defects on a tubular structure thereof, said deformities or said defects comprising bifurcations, curvatures, elbows, constrictions, dilatations;
said scaffold consisting of electrospun silk fibroin, copolymers of polyglycolic acid/polylactic acid (PGA/PLA) or copolymers of polyglycolic acid/polycaprolactone (PGA/PCL).

2. The in vitro model according to claim 1, wherein said vascular structure is selected from among blood vessels or blood ducts or central or peripheral circulatory system valves.

3. The in vitro model according to claim 2, wherein said vascular structure is selected from among arteries, veins, capillaries, aortic or mitral valve.

4. The in vitro model according to any one of claim 1, wherein said deformities or said defects of the tubular structure comprise combinations of bifurcations, curvatures, elbows, constrictions, dilatations.

5. A method for testing a medical device or a drug so as to verify the effectiveness and safety thereof before an in-vivo use thereof on the man or animal, said method comprising the following steps:
preparing a substantially tubular-shaped scaffold having the dysfunctional anatomical and physiological characteristics suitable to simulate a damage or a deformation or a deterioration due to an aneurysm, stenosis, sclerosis plaques, forms of tumours or cardiomyopathies, said scaffold having deformities or defects on the tubular structure thereof which are bifurcations, curvatures, elbows, constrictions, dilatations;
seeding at least one part of the interior lumen of said scaffold with endothelial cell lines so as to obtain a continuous and homogeneous layer of seeded endothelial cells (seeding method), optionally seeding at least one part of the outer surface of said scaffold with muscle cell lines;
promoting the growth of said endothelial cells, and optionally said muscle cells, up to obtaining a continuous and uniform layer of endothelial cells, to obtain said in vitro model;
introducing into said in vitro model a medical device or a drug subject of test, and
allowing the circulation (perfusion model) in said in-vitro model comprising said medical device or drug of a human whole blood sample, artificial blood or derivatives thereof so as to evaluate the behaviour and the interaction of said medical device or drug with said human whole blood sample, artificial blood or derivatives thereof.

6. A method or process for the production of an engineered vascular tissue or construct, preferably a scaffold (21) having a lumen covered with functional and continuous endothelium having a confluent cell monolayer, for testing medical or veterinarian products, said process comprising applying:
a method for seeding an endothelial cell culture in the lumen of a scaffold (21) to obtain a seeded scaffold (21); said seeded scaffold (21) being present in a bioreactor (11), to obtain a bioreactor (11)-seeded scaffold (21) system;
wherein said seeding method comprises the steps of:
releasing said endothelial cell culture in form of a cell suspension comprising a fresh growth medium and endothelial cells in a container (91) mounted on a T-shaped connector (T2) arranged upstream of the bioreactor (11) by means of a rotary connector (CR1); followed by
releasing said endothelial cell culture in the lumen of the scaffold (21) present in the bioreactor chamber (11) with a continuous flow such that the flow speed allows said cell suspension to drip into the T-shaped connector (T2) without generating air bubbles and pushing the air bubbles present in the lumen of the scaffold (21) towards an opening of a T-shaped connector (T3) arranged downstream of the bioreactor (11) allowing the outflow thereof;
and, subsequently,
a method for perfusion—with a fresh growth medium having a temperature comprised in the range between 30° C. and 45° C., preferably at 37° C.—of the endothelial cells present in the lumen of said seeded scaffold (21); said perfusion method being obtained by connecting a perfusion circuit (51-56) or (51-57 and BT) to said bioreactor (11)-seeded scaffold (21) system;
wherein said perfusion method comprises a step of
partly filling an element for removing the air bubbles (71) or (BT) present in the perfusion circuit with said fresh growth medium, wherein said element for removing the air bubbles (71) or (BT) comprises a chamber, a cap that closes said chamber, an access with inflow function (211) and an access with outflow function (212), wherein said chamber of the element for removing the air bubbles (71 or BT) has a volume and wherein a first part of said volume is filled with said fresh growth medium and wherein a second part of said volume is filled with air, said second part of said volume having the function of trapping the air bubbles present in said fresh growth medium which flows through said access with inflow function (211) and said access with outflow function (212).

7. The process according to claim 5, wherein said method for seeding said endothelial cell culture in the lumen of said scaffold (21) comprises:
mounting the scaffold (21), preferably an electrospun silk fibroin tubular scaffold, on the grips of a scaffold-holder (13, 13a, 13b) and housing said scaffold-holder (13, 13a, 13b) with the scaffold (21) in the bioreactor chamber (11), to obtain a bioreactor(11)-scaffold (21) system;
followed by
injecting the fresh growth medium into the lumen of said scaffold (21) fixed on said scaffold-holder (13) arranged inside the bioreactor chamber (11); followed by
adding said fresh growth medium into the bioreactor chamber (11) where said scaffold-holder (13, 13a, 13b) with the scaffold (21) is present injected with said growth medium; followed by
leaving for a time interval comprised between 1 hour and 18 hours at a temperature comprised between 20° C. and 30° C., preferably 25° C., said growth medium in the lumen of the scaffold (21) and in the bioreactor chamber (11) where said scaffold-holder (13) with the scaffold (21) is present injected with said growth medium; followed by
clearing the internal of the lumen of the scaffold (21) and of the bioreactor chamber (11) of the growth medium; followed by
releasing said endothelial cell culture in said container (91), preferably said container (91) is a syringe; followed by
releasing said cell suspension in the lumen of the scaffold (21); followed by
adding said fresh growth medium in the bioreactor chamber (11) where said scaffold-holder (13) with the scaffold (21) is present seeded containing said cell suspension in the lumen; and followed by
incubating, preferably for 24 hours at 37° C. in presence of 5% of CO₂, the scaffold (21) housed in the bioreactor chamber (11).

8. The process according to claim 5, wherein said method for the perfusion of the endothelial cells present in the lumen of said seeded scaffold (21) comprises:
preparing said closed perfusion circuit comprising the tubes (51), (52), (53), (54), and, optionally, (55);
occluding the tube (54) or (55) of the perfusion circuit using a closing element (171) in a position proximal to a connector (C), preferably said closing element is a clamp or the like; followed by
unscrewing the connector (C) arranged between the tube (53) or (54) and the tube (54) or (55) respectively in the perfusion circuit;
screwing the tube (53) or (54) of the perfusion circuit to an open lateral end of the T-shaped connector (T2) upstream of the bioreactor (11) at a lateral access thereof; followed by
opening the T-shaped connector (T3) downstream of the bioreactor (11) and unscrewing a cap of a lateral opening of the T-shaped connector (T3); followed by
connecting the tube (54) or (55) of the perfusion circuit to the lateral opening of the T-shaped connector (T3) arranged downstream of the bioreactor (11) and removing the closing element (171);
followed, if need be, by
inserting—between the tube (53) and the under-pump tube (52) of the perfusion circuit—the element for removing the air bubbles (71).

9. The process according to claim 6, wherein the element for removing the air bubbles (71) or (BT) is a bubble-trap or the like.

10. The process according to claim 5, wherein the scaffold (21), preferably a tubular scaffold, is selected from among polymeric scaffolds of synthetic or natural origin, wherein said polymeric scaffolds are formed by only one polymer or by copolymers, preferably electro spun silk fibroin or copolymers of polyglycolic acid/polylactic acid (PGA/PLA) or copolymers of polyglycolic acid/polycaprolactone (PGA/PCL).

11. The process according to claim 5, wherein the endothelial cells are selected from among the cells that form an endothelium of a vascular tissue, preferably HAOECs (human aortic endothelial cells), HCAECs (human coronary artery endothelial cells), HMEVECs (human dermal microvascular endothelial cells) or HUVECs (human umbilical vein endothelial cells).

12. The process according to claim 6, wherein the growth medium used is the Endothelial Growth Medium comprising fetal bovine serum (2%), adenine (0.2 μg/ml), ammonium metavanadate (0.0006 μg/ml), amphotericin B (0.3 μg/ml), calcium chloride 2H₂O (300 μg/ml), choline chloride (20 μg/ml), copper sulphate 5H₂O (0.002 μg/ml), trioptic acid DL-6,8 (0.003 μg/ml), folinic acid (calcium) (0.6 μg/ml), heparin (4 μg/ml), hydrocortisone (2 μg/ml), L-aspartic acid (15 μg/ml), L-cysteine (30 μg/ml), L-tyrosine (20 μg/ml), manganese sulphate monohydrate (0.0002 μg/ml), ammonium molybdate 4H₂O (0.004 μg/ml), nicotinamide (8 μg/ml), nickel chloride 6H₂O (0.0001 μg/ml), penicillin (60 μg/ml), phenol red sodium salt (15 μg/ml), potassium chloride (300 μg/ml), putrescine dihydrochloride (0.0002 μg/ml), pyridoxine hydrochloride (3 μg/ml), sodium metasilicate 9H₂O (3 μg/ml), sodium sulphate 7H₂O (200 μg/ml), sodium selenite (0.01 μg/ml), streptomycin sulphate (100 μg/ml), thiamine hydrochloride (4 μg/ml) and zinc sulphate 7H₂O (0.0003 μg/ml), preferably heated to 37° C.

13. A scaffold (21) for use in the model of claim 1, the scaffold having a lumen coated with a functional and continuous endothelium (21) having a confluent cell monolayer obtained by means of a process comprising the following steps:
preparing a substantially tubular-shaped scaffold having the dysfunctional anatomical and physiological characteristics suitable to simulate a damage or a deformation or a deterioration due to an aneurysm, stenosis, sclerosis plaques, forms of tumours or cardiomyopathies;
seeding at least one part of the interior lumen of said scaffold with endothelial cell lines so as to obtain a continuous and homogeneous layer of seeded endothelial cells (seeding method), optionally seeding at least one part of the outer surface of said scaffold with muscle cell lines;
promoting the growth of said endothelial cells, and optionally said muscle cells, up to obtaining a continuous and uniform layer of endothelial cells, to obtain said in vitro model.

14. Use of the scaffold (21) according to claim 1, for conducting in vitro preclinical or clinical tests of a medicinal product for human use or of a veterinarian product for animal use to be used in the cardiovascular and peripheral vascular region, preferably valves, heart valves, stents, grafts, catheters, bandages or nets.
